(19) United States
(12) Patent Application Publication
(10) Pub. No.: US 2023/0001009 A1
(43) Pub. Date: Jan. 5, 2023

(54) NANOPARTICLE COMPOSITIONS AND METHODS OF USE

(71) Applicant: The Trustees of the University of Pennsylvania, Philadelphia, PA (US)

(72) Inventors: Jacob Myerson, Clayton, MO (US); Jacob Brenner, Princeton Junction, NJ (US); Vladimir Muzykantov, Bryn Athyn, PA (US); Landis R. Walsh, Winchester, MA (US); Priyal Patel, Philadelphia, PA (US)

(21) Appl. No.: 17/781,945

(22) PCT Filed: Dec. 3, 2020
(86) PCT No.: PCT/US2020/063120

§ 371 Date: Jun. 2, 2022

Related U.S. Application Data
(60) Provisional application No. 62/943,469, filed on Dec. 4, 2019.

(51) Int. Cl. A61K 47/69; A61K 47/42; A61K 47/62; A61K 9/00; A61K 9/127

(52) U.S. Cl. CPC A61K 47/6913; A61K 47/42; A61K 47/62; A61K 9/0019; A61K 9/1272

(57) ABSTRACT

Provided herein are compositions comprising a nanoparticle and a D20 tag. The D20 tag comprises dibenzocyclooctyne (DBCO) covalently attached to a protein. Also provided are diagnostic and therapeutic methods utilizing the nanoparticle composition.

### STATEMENT REGARDING FEDERALLY SPONSORED RESEARCH OR DEVELOPMENT

This invention was made with government support under Grant Nos. HL125462-04 and 1K08HL138269-01 awarded by the National Institutes of Health. The government has certain rights in this invention.

### BACKGROUND OF THE INVENTION

Neutrophils play an integral role in the progression of acute inflammatory damage arising from a variety of diseases. Neutrophils can be activated by a variety of initiating factors, including chemokine release from platelets or endothelial cells, interactions with bacterial lipopolysaccharides (LPS), or damage-associated molecular patterns (DAMPs). Targeted drug delivery and imaging contrast focused on neutrophils may be an attractive route for nanomedicines designed for treatment and diagnosis of acute inflammatory disorders.

Activated neutrophils are particularly important in the etiology of lung disease culminating in acute respiratory distress (ARDS), a disease affecting ˜200,000 American patients per year with a ˜35-50% mortality rate. Neutrophils are retained in the lungs at high concentrations under naïve conditions, but adhere to the lung vasculature even more avidly after acute systemic inflammatory insult. In ARDS, systemic or pulmonary inflammation results in extravasation of activated neutrophils in the lung vasculature, leading to disruption of the endothelial barrier and accumulation of neutrophils and edematous fluid in the air space of the lungs.

Focusing treatment or diagnostic strategies on neutrophils remains an open problem. Antibodies against markers such as Ly6G can achieve targeting to neutrophils, but also dramatically affect neutrophil function. Therefore, antibody targeting strategies have not been widely adopted for targeted drug delivery to neutrophils. Two previous studies noted that activated neutrophils take up denatured and agglutinated bovine albumin, concluding that denatured protein was the critical factor in the neutrophil-particle interactions.

Indeed, nanoparticle structure and composition can affect biodistribution and targeting behaviors, competing with and even superseding targeting functions defined by engineered surface chemistries (e.g., antibody functionalization). Shape, size, deformability, and zeta potential have been cited as engineering parameters that help define pharmacokinetics and immune interactions of nanoparticle drug carriers. Engineering of nanoparticle structure, rather than engineering of antibody- or peptide-based surface chemistry, shapes the in vivo behavior of translational nanomedicines like Abraxane, Doxil, and Onpattro.

A continuing need in the art exists for new and effective tools and methods for targeting leukocytes, including neutrophils.

### SUMMARY OF THE INVENTION

The needs of the art are met in the methods and compositions disclosed herein. In one embodiment, these methods and compositions provide an advantage of delivering drugs via the intravascular route to the lungs, shuttling potentially multiple drugs to the inflamed alveoli. In other embodiment, similar vascular drug delivery methods and compositions are provided which permit intravascular drug delivery for other diseases or to other inflamed or injured tissues. IN certain embodiments, thee compositions described herein provide a drug carrier that can massively increase drug concentration in an organ in a manner independent of antibodies.

In one aspect, provided herein is a composition comprising a nanoparticle and a D20 tag. The D20 tag comprises dibenzocyclooctyne (DBCO) covalently attached to a protein.

In another aspect, a method of generating a nanoparticle composition is provided. The method includes conjugating DBCO to a protein to generate a D20 tag. In certain embodiments, the nanoparticle is a liposome. In certain embodiments, the nanoparticle is a lipid nanoparticle (LNP). In certain embodiments, the nanoparticle is a protein-based nanoparticle.

In another aspect, provided herein is a method of generating a composition comprising a nanoparticle having a D20 tag comprising dibenzocyclooctyne (DBCO) covalently attached to a protein, the method comprising conjugating DBCO to the protein to generate the D20 tag.

In yet another aspect, a method of treating lung injury in a subject in need thereof is provided. The method includes administering the nanoparticle composition as described herein to a subject. In certain embodiments, the subject has ARDS, sepsis, or pneumonia.

In another aspect, a method of targeting leukocytes is provided. In certain embodiments, the method includes administering the nanoparticle composition as described herein to a subject. The leukocytes may be neutrophils, monocytes, macrophages, eosinophils, basophils, NK cells, lymphocytes, or dendritic cells. In yet a further embodiment, the leukocytes are marginated and/or present in the lung of the subject

In yet another aspect, a method of treating an inflamed tissue in a subject in need thereof is provided. The method includes administering the nanoparticle composition as described herein to a subject. In certain embodiments, the subject has a subacute or acute infection and/or subacute or acute inflammatory condition.

In another aspect, the use of a nanoparticle composition provided in the treatment of a subject having an injury or inflammation in the lung or other tissue is provided.

Still other aspects and advantages of these compositions and methods are described further in the following detailed description of the preferred embodiments thereof.

### BRIEF DESCRIPTION OF THE DRAWINGS

FIG. 1A-FIG. 1E show neutrophil accumulation in acutely inflamed pulmonary vasculature.

FIG. 2A-FIG. 2K show lysozyme-dextran nanogels and crosslinked albumin nanoparticles accumulate in neutrophils in inflamed lungs.

FIG. 3A-FIG. 3E show uptake of different nanoparticles in naïve and IV-LPS-inflamed Lungs.

FIG. 4A-FIG. 4E show engineering of liposome surface chemistry to confer liposome specificity for neutrophils in LPS-inflamed lungs.

FIG. 5A-FIG. 5D show specificity of lysozyme-dextran nanogels for LPS-inflamed lungs vs. edematous lungs and SPECT imaging of lysozyme-dextran nanogels in LPS-inflamed lungs.

FIG. 6A-FIG. 6G show therapeutic effects of neutrophil-targeted liposomes in model acute respiratory distress.

FIG. 7A-FIG. 7E show uptake of lysozyme-dextran nanogels in ex vivo human lungs.

FIG. 8A-FIG. 8D show dynamic light scattering characterization of tested nanoparticles.

FIG. 9A-FIG. 9B show flow cytometric characterization of lysozyme-dextran nanogel uptake in naïve and inflamed lungs.

FIG. 10A-FIG. 10E show flow cytometric characterization of crosslinked albumin nanoparticle uptake in leukocytes in naïve and inflamed lungs.

FIG. 11A-FIG. 11D show pharmacokinetics of lysozyme-dextran nanogels in naïve and IV-LPS-injured mice.

FIG. 12 shows biodistributions of lysozyme-dextran nanogels in naïve and intratracheal LPS-injured mice.

FIG. 13A-FIG. 13B show biodistributions of lysozyme-dextran nanogels after footpad administration of LPS.

FIG. 14A-FIG. 14D show circular dichroism spectroscopic characterization of protein secondary structure and ANSA characterization of hydrophobic domain accessibility for lysozyme-dextran nanogels and crosslinked albumin nanoparticles.

FIG. 15 shows biodistributions of structural variants of lysozyme-dextran nanogels in naïve and IV-LPS-injured mice.

FIG. 16 shows biodistributions of structural and compositional variants of crosslinked protein nanoparticles in naïve and IV-LPS-injured mice.

FIG. 17 shows biodistributions of compositional variants of charge-agglutinated green fluorescent protein nanoparticles in naïve and IV-LPS-injured mice.

FIG. 18 show biodistributions of adenovirus, adeno-associated virus, and horse spleen ferritin nanocages in naïve and IV-LPS-injured mice.

FIG. 19 shows biodistributions of bare liposomes and IgG-coated polystyrene nanoparticles in naïve and IV-LPS-injured mice.

FIG. 20 shows biodistributions of isolated albumin, lysozyme, and transferrin in naïve and IV-LPS-injured mice.

FIG. 21 shows biodistributions in naïve mice for bare liposomes, liposomes conjugated to IgG via SATA-maleimide reaction, and liposomes conjugated to IgG via DBCO-azide reaction.

FIG. 22 shows biodistributions of DBCO:IgG (20:1) liposomes in mice 1, 2, and 6 hours after intratracheal LPS injury.

FIG. 23A-FIG. 23B show spectrophotometric characterization of DBCO conjugation to IgG.

FIG. 24A-FIG. 24G show flow cytometric characterization of DBCO:IgG (20:1) liposome uptake in leukocytes and endothelial cells in naïve and inflamed lungs.

FIG. 25 shows biodistributions of isolated DBCO:IgG (20:1) in naïve and IV-LPS-injured mice.

FIG. 26A-FIG. 26B show circular dichroism spectroscopic characterization of protein secondary structure in DBCO-modified IgG and ANSA characterization of hydrophobic domain accessibility on DBCO:IgG (20:1) liposomes.

FIG. 27 shows quantification of CT attenuation in edematous and naïve mouse lungs.

FIG. 28 shows lysozyme-dextran nanogel and ferritin nanocage uptake in human lungs as a function of tissue perfusion.

FIG. 29 shows results from altering cholesterol content in D20-IgG liposomes. Liposomes with 40% cholesterol accumulate in the injured lungs at 1.87-fold higher concentration than 25% cholesterol liposomes.

FIG. 30 shows results using DBCO-tagged bovine albumin (BSA) liposomes. A 2.95-fold increase in uptake in injured mouse lungs was observed (relative to levels obtained with bare liposomes).

FIG. 31A-FIG. 31F show complement opsonization of nanoparticles with agglutinated protein is necessary for their uptake in neutrophils. (FIG. 31A) Flow cytometry data indicating neutrophils take up lysozyme-dextran nanogels (NGs) after NG incubation in mouse serum (n=18) but not after NGs incubation in buffer (n=18) (*=p<0.01). (FIG. 31B) Mass spectrometry characterization of proteins adsorbed on NGs after incubation with mouse serum as in FIG. 31A. Plotted data indicates the concentration of detected peptides associated with the five most abundant proteins on the serum-incubated NGs (n=3 serum/NG preparations). (FIG. 31C and FIG. 31D) Flow cytometric assessment of NG uptake in mouse neutrophils after NG incubation with buffer, mouse serum, heat-treated mouse serum, and mouse serum treated with cobra venom factor (CVF) to specifically deplete complement. Example histograms of NG fluorescence in neutrophils for different serum conditions are depicted in FIG. 31C. Data reflecting NG mean fluorescence in neutrophils for different serum conditions is plotted in FIG. 31D. In FIG. 31D, bars indicate naïve neutrophils (n=18 naïve serum, n=10 heat-treated serum, n=11 CVF-treated serum) and LPS-stimulated neutrophils (n=12 naïve serum, n=5 heat-treated serum, n=7 CVF-treated serum) (*=p<0.01 relative to naïve serum-incubated values). (FIG. 31E) In vivo biodistributions of NGs in naïve mice, mice treated with CVF, mice treated with intravenous LPS, and mice treated with intravenous LPS and CVF (n=4 for all groups, *=p<0.01 relative to intravenous LPS alone). (FIG. 31F) Mass spectrometry characterization of complement opsonization of NGs (n=3) and human adenovirus (n=3). Peptide count data as in FIG. 31B is normalized to peptide counts on NGs incubated with complement-depleted CVF-treated serum. Relative complement quantities below zero indicate complement opsonization at lower levels than on NGs after treatment with complement-depleted serum.

FIG. 32A-FIG. 32C show proteomics characterization of serum opsonization of lysozyme-dextran nanogels and human adenovirus. (FIG. 32A) Peptide counts from mass spectrometry data indicating the ten most abundant proteins identified on the surface of lysozyme-dextran nanogels after incubation with mouse serum, with quantities of the same proteins on human adenovirus capsids included for comparison. (FIG. 32B) Peptide counts from mass spectrometry data indicating the ten most abundant proteins identified on the surface of human adenovirus capsids after incubation with mouse serum, with quantities of the same proteins on lysozyme-dextran nanogels included for comparison. (FIG. 32C) Peptide counts indicating mass spectrometry quantification of complement proteins on the surface on lysozyme-dextran nanogels and human adenovirus capsids after incubation with mouse serum. Peptide counts on lysozyme-dextran nanogels after incubation with complement-depleted cobra venom factor (CVF)-treated mouse serum. Insets: Mass spectrometry measurement of abundance of corresponding proteins in serum preparations. (*=p<0.01)

FIG. 33A-FIG. 33D show flow cytometric characterization of lysozyme-dextran nanogel uptake in neutrophils in vitro under different serum conditions. (FIG. 33A) Gating strategies indicating determination of lysozyme-dextran nanogel fluorescence vs. levels of anti-Ly6G neutrophil staining after treatment of lysozyme-dextran nanogels with different serum conditions. (FIG. 33B and FIG. 33C) Example histograms of lysozyme-dextran nanogel fluorescence in naïve (FIG. 33B) and LPS-stimulated (FIG. 33C) neutrophils with lysozyme-dextran nanogels treated with different serum conditions. (FIG. 33D) Quantification of mean lysozyme-dextran nanogel fluorescence intensity in neutrophils after treatment of lysozyme-dextran nanogels with different serum conditions. Data is as in FIG. 31C and FIG. 31D, with data incorporating serum from CVF-treated mice added.

FIG. 34 shows biodistributions of lysozyme-dextran nanogels in mice treated with cobra venom factor and/or intravenous LPS. In vivo biodistributions of NGs in naïve mice, mice treated with CVF, mice treated with intravenous LPS, and mice treated with intravenous LPS and CVF. Data is as in FIG. 31E, with addition of heart, kidneys and lung:blood values (*=p<0.01 relative to intravenous LPS alone).

FIG. 35A-FIG. 35J shows effects of nanoparticles with agglutinated protein in model acute respiratory distress syndrome (ARDS). (FIG. 35A) Timeline: Nanoparticles or vehicle were administered as an intravenous bolus two hours after nebulized LPS administration. Bronchoalveolar lavage (BAL) fluid was harvested 22 hours after liposome or vehicle administration. (FIG. 35B) Concentration of protein in BAL fluid, reflecting quantity of edema, with and without treatment with different nanoparticles. Quantities are represented as degree of protection against infiltration into alveoli, as extrapolated from levels in naïve mice (100% protection) and untreated mice with LPS-induced injury (0% protection). (FIG. 35C) Concentration of leukocytes in BAL fluid, represented as degree of protection as in (FIG. 35B). Data for nanoparticle-treated mice in FIG. 35B and FIG. 35C indicate edema and leukocyte leakage 22 hours after treatment with 30 mg/kg of the indicated nanoparticles (*=p<0.01). (FIG. 35D and FIG. 35E) Dose-response for edema (FIG. 35D) and leukocyte infiltration (FIG. 35E) in alveoli of LPS-injured mice treated with DBCO-IgG liposomes. Data were obtained as in FIG. 35B and FIG. 35C, but with different liposome doses. (FIG. 35F) Chemokine CXCL2 levels in alveoli of LPS-injured mice with and without DBCO-IgG liposome treatment. Dashed line indicates CXCL2 levels in alveoli of naïve mice. (‡=p<0.05). (FIG. 35G) Concentration of neutrophils in BAL fluid, represented as protection against infiltration into BAL, as in FIG. 35B and FIG. 35C, with and without dosing with 30 mg/kg DBCO-IgG liposomes (*=p<0.01). (FIG. 35H) Biodistributions of anti-Ly6G antibody in naïve mice (n=3), LPS-injured mice (n=3), and mice treated with 10 mg/kg DBCO-IgG liposomes, with organs sampled at 1 hour after treatment (n=3) or 22 hours after treatment (n=3) (*=p<0.01 comparison to untreated LPS-inflamed data). (FIG. 35I) Complete blood count analysis of circulating leukocyte concentrations in naïve mice (n=3), LPS-injured mice (n=3), and mice treated with 10 mg/kg DBCO-IgG liposomes, with blood sampled 22 hours after treatment (n=3) (*=p<0.01 comparison to untreated LPS-inflamed data). (FIG. 35J) Schematic for the fate of neutrophils in mice with model ARDS, with and without DBCO-IgG liposome treatment, based on data in FIG. 35G-FIG. 35I.

FIG. 36A-FIG. 36B show raw quantification of pulmonary edema and leukocyte leak into alveoli in model ARDS with different nanoparticle treatments. Data as in FIG. 35A and FIG. 35B, represented in terms of raw quantity of protein (FIG. 36A) and leukocyte accumulation (FIG. 36B) in alveoli under naïve, injured, and different nanoparticle treatment conditions.

FIG. 37A-FIG. 37B show dose-responses for raw quantification of pulmonary edema and leukocyte leak into alveoli in model ARDS with DBCO-IgG liposome treatment. Data as in FIG. 35C and FIG. 35D, represented in terms of raw quantity of protein (FIG. 37A) and leukocyte accumulation (FIG. 37B) in alveoli after treatment with different doses of DBCO-IgG liposomes.

FIG. 38A-FIG. 38D show dose-responses for CXCL2 concentration in bronchoalveolar lavage fluid, lung tissue, plasma, and liver tissue after DBCO-IgG liposome treatment in model ARDS. Chemokine CXCL2 levels in BAL fluid (FIG. 38A), lung tissue (FIG. 38D), plasma (FIG. 38C), and liver tissue (FIG. 38D) of LPS-injured mice with and without DBCO-IgG liposome treatment. Dashed line indicates CXCL2 levels in naïve mice. Data in FIG. 38A is equivalent to data in FIG. 35E (‡=p<0.05).

FIG. 39A-FIG. 39D show dose-responses for IL-6 concentration in bronchoalveolar lavage fluid, lung tissue, plasma, and liver tissue after DBCO-IgG liposome treatment in model ARDS. Cytokine IL-6 levels in BAL fluid (FIG. 39A), lung tissue (FIG. 39D), plasma (FIG. 39C), and liver tissue (FIG. 39D) of LPS-injured mice with and without DBCO-IgG liposome treatment (‡=p<0.05).

FIG. 40A-FIG. 40D show quantification of neutrophil leak into alveoli in model ARDS with different nanoparticle treatments. (FIG. 40A and FIG. 40B) Concentration of neutrophils in BAL fluid, with and without treatment with different nanoparticles. In FIG. 40A, quantities are represented as degree of protection against neutrophil infiltration into alveoli, as extrapolated from levels in naïve mice (100% protection) and untreated mice with LPS-induced injury (0% protection). (FIG. 40B) Data as in FIG. 40A, represented as raw quantities of neutrophils in alveoli. (FIG. 40C and FIG. 40D) Dose-response for neutrophil infiltration in alveoli of LPS-injured mice treated with different doses of DBCO-IgG liposomes. Data in FIG. 40C represent protection against neutrophil infiltration and data in FIG. 40D represent raw quantities of neutrophils in the alveoli.

FIG. 41A-FIG. 41B show intravascular neutrophil tracing in mice after DBCO-IgG liposome treatment. (FIG. 41A) Representation of data as in FIG. 35G. (FIG. 41B) Tracing of anti-Ly6G neutrophil antibody in sham-injured liposome-treated mice for comparison.

FIG. 42 shows dose-response for weight change over the course of model ARDS in mice treated with DBCO-IgG liposomes. Data indicate no significant change from untreated values for all tested doses of DBCO-IgG liposomes.

FIG. 43A-FIG. 43B show complete blood count analysis assessment of circulating leukocyte concentrations and size distributions in mice treated with DBCO-IgG liposomes and/or LPS. (FIG. 43A) Complete blood count analysis data indicating circulating leukocyte size distributions in naïve mice, LPS-injured mice, mice treated with 2.5 mg/kg DBCO-IgG liposomes, mice treated with 5 mg/kg DBCO-IgG liposomes, and mice treated with 10 mg/kg DBCO-IgG liposomes. Blood was sampled 22 hours after liposome treatment and 24 hours after induction of LPS injury. Leftmost peak indicates circulating lymphocytes and rightmost peak indicates circulating neutrophils. (FIG. 43B) Complete blood count quantification of circulating leukocyte concentrations in naïve mice (first column), LPS-injured mice (second column), mice treated with 2.5 mg/kg DBCO-IgG liposomes (third column), mice treated with 5 mg/kg DBCO-IgG liposomes (fourth column), and mice treated with 10 mg/kg DBCO-IgG liposomes (fifth column). Data for 10 mg/kg liposome dose and controls are as in FIG. 35H. (*=p<0.01 comparison to untreated LPS-inflamed data)

FIG. 44A-FIG. 44C show complete blood count analysis of circulating platelet and red blood cell concentrations and size distributions in mice treated with DBCO-IgG liposomes and/or LPS. (FIG. 44A) Complete blood count analysis data indicating circulating platelet (leftmost peak) and red cell (larger rightmost peak) size distributions in naïve mice, LPS-injured mice, mice treated with 2.5 mg/kg DBCO-IgG liposomes, mice treated with 5 mg/kg DBCO-IgG liposomes, and mice treated with 10 mg/kg DBCO-IgG liposomes. Blood was sampled 22 hours after liposome treatment and 24 hours after induction of LPS injury. (FIG. 44B) Complete blood count quantification of circulating red cell concentrations and properties in naïve mice, LPS-injured mice (n=3), mice treated with 2.5 mg/kg DBCO-IgG liposomes, mice treated with 5 mg/kg DBCO-IgG liposomes, and mice treated with 10 mg/kg DBCO-IgG liposomes. RBC=red blood cell concentration, HGB=hemoglobin concentration, HCT=red blood cell hematocrit, MCV=mean red blood cell volume, MCH=mean red blood cell hemoglobin content, MCGH=mean red blood cell hemoglobin concentration, RDWc=width of the red blood cell size distribution. (FIG. 44C) Platelet concentrations and properties for the same experimental groups as depicted in FIG. 44B. PLT=platelet concentration, PCT=platelet hematocrit, MPV=mean platelet volume, PDWc=width of the platelet size distribution.

FIG. 45A-FIG. 45C show biodistribution of lysozyme-dextran nanogels in a localized footpad inflammation model. (FIG. 45A) Complete Freund's adjuvant (CFA) was injected into one hindlimb footpad six hours prior to nanoparticle tracing experiments. Inflammation in CFA-injected feet (ipsilateral hindpaw) was evident via ˜75% increase in lateral paw thickness, relative to saline sham control and contralateral paw. (FIG. 45B) Lysozyme-dextran nanogels were traced in mice with CFA injury. Nanogels accumulated in the injured hindpaw at ˜2.5-fold greater concentrations than in sham-injected and contralateral paws. (FIG. 45C) CFA induced no significant differences in nanogel accumulation in other organs.

FIG. 46A-FIG. 46F show flow cytometric characterization of lysozyme-dextran nanogel uptake in different cell types in inflamed and naïve footpads. Feet injected with complete Freund's adjuvant (CFA) or saline shame were disaggregated after intravenous administration of fluorescent lysozyme-dextran nanogels and resultant single cell suspensions were analyzed with flow cytometry. (FIG. 46A) Plots of CD45/leukocyte-associated fluorescence against lysozyme-dextran nanogel fluorescence in sham-injured and CFA-injured feet. (FIG. 46B) Analysis according to the quadrant gates depicted in FIG. 46A determined a ˜3-fold increase in the number of leukocytes in CFA-injured feet and showed that ˜90% of nanogel uptake in both CFA-injured and sham-injured feet was attributable to leukocytes. (FIG. 46C) Plots of F480/macrophage/monocyte-associated fluorescence against lysozyme-dextran nanogel fluorescence in sham-injured and CFA-injured feet. (FIG. 46D) Analysis of monocyte/macrophage-nanogel association according to the gates depicted in FIG. 46C. Macrophages form a negligible fraction of the leukocyte infiltrates in injured paws and have minimal role in uptake of nanogels in the inflamed feet. (FIG. 46E) Plots of Ly6G/neutrophil-associated fluorescence against lysozyme-dextran nanogel fluorescence in sham-injured and CFA-injured feet. (FIG. 46F) Analysis of neutrophil-nanogel association according to the gates depicted in FIG. 46E. The number of neutrophils and the quantity of nanogel signal associated with neutrophils significantly increased (˜3-fold) in CFA-injured feet, relative to sham injury. Inset: histogram of nanogel fluorescence in neutrophils in sham- and CFA-injured feet.

FIG. 47 shows the effect of cholesterol content on liposome uptake by neutrophils. Mice received IV LPS as a model of sepsis/ARDS and were injected five hours later with liposomes (approx. 5 mg/kg). Lung uptake provides a measure of tropism to marginated neutrophils. D5=5 DBCO per protein (IgG) on the tag. D5=5 DBCO per IgG on the tag. D20=20 per IgG on the tag. 25% chol=25% (moles cholesterol/total moles of lipid) of the lipids in liposomes was cholesterol. 25% cholesterol=25% (moles cholesterol/total moles of lipid) of the lipids in liposomes was cholesterol. Neutrophil tropism was improved with D20 and 40% cholesterol.

FIG. 48 shows the effect of the number of D20 tags on liposome uptake by neutrophils. Mice received IV LPS as a model of sepsis/ARDS and were injected five hours later with liposomes. Lung uptake provides a measure of tropism to marginated neutrophils. Liposomes had a D20 tag (20 DBCO per protein, here IgG) and 200, 150, 100, 50, or 25 D20-IgG molecules per liposome. There was a marked improvement in uptake when there were >100 molecules per liposome.

### DETAILED DESCRIPTION

The nanoparticle compositions and methods described herein have been shown to be useful in preferentially targeting leukocytes, more specifically, in some embodiments, neutrophils. The compositions and methods are useful in treating acute inflammatory conditions, including lung injury and the like. As demonstrated herein, nanoparticle structural properties that shape interactions with neutrophils in the setting of acute inflammation were studied. Due to the key role of neutrophils in lung physiology and the pathology of acute lung disease (noted above for its broad clinical impact) and the high concentration of neutrophils in the lung vasculature, the localization of nanoparticles to the lung vasculature in LPS injury models was of particular interest in the studies described.

Provided herein, in one aspect, is a composition comprising a nanoparticle and a D20 tag. The nanoparticle is covalently attached to the D20 tag. By “nanoparticle” (also referred to as “nanocarrier” or “NP”) as used herein is meant a particle having diameter of between 1 to 1000 nm. The terms nanoparticle, nanocarrier, liposome, and LNP may be used interchangeably. In one embodiment the NP is globular. Inclusive in this definition are particles with a diameter of at least 1, at least 20, at least 40, at least 60, at least 80, at least 100, at least 120, at least 140, at least 160, at least 180, at least 200, at least 220, at least 240, at least 260, at least 280, at least 300 nm in diameter. In other embodiments, also included are particles having diameters of at least 320, at least 340, at least 360, at least 380, at least 400, at least 420, at least 440, at least 460, at least 480, at least 500, at least 520, at least 540, at least 560, at least 580, at least 600, at least 620, at least 640, at least 660, at least 680, at least 700 nm. In yet other embodiment, also included are particles having diameters of at least 720, at least 740, at least 760, at least 780, at least 800, at least 820, at least 840, at least 860, at least 880, at least 900, 920, at least 940, at least 960, at least 980, up to at least 1000 nm. All numbers and fractions between any two of these numbers are also included.

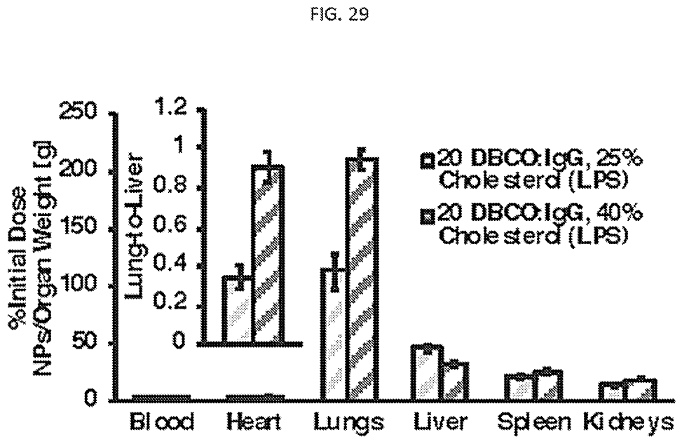

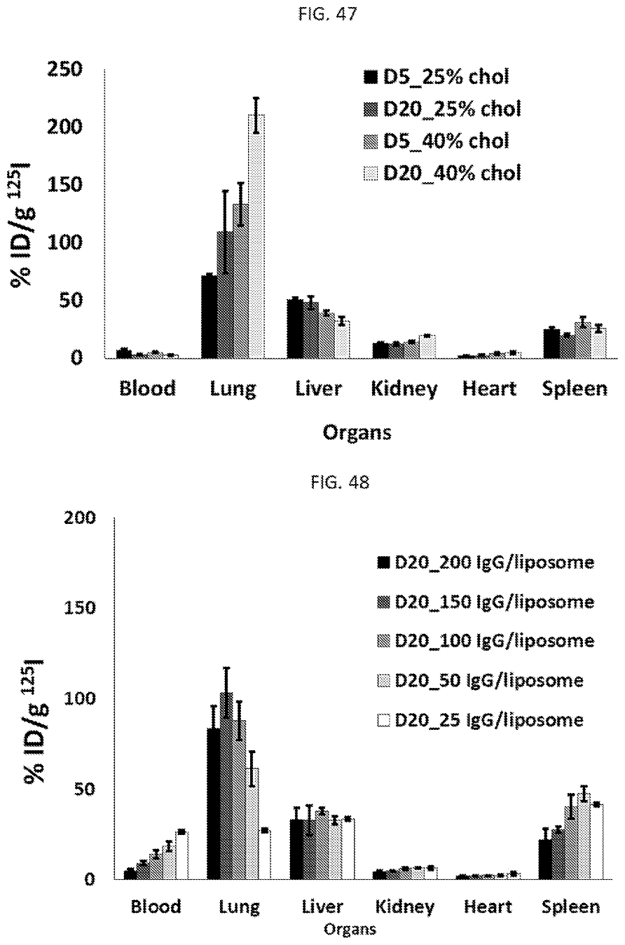

In one aspect, the nanoparticle employed herein is a liposome. By “liposome” as used herein is meant a microscopic spherical particle formed by a lipid bilayer enclosing an aqueous compartment. Liposomes can be created from cholesterol and natural non-toxic phospholipids. Due to their size and hydrophobic and hydrophilic character, liposomes are promising systems for drug delivery. Liposome properties differ considerably with lipid composition, surface charge, size, and the method of preparation. Furthermore, the choice of bilayer components determines the ‘rigidity’ or ‘fluidity’ and the charge of the bilayer. For instance, unsaturated phosphatidylcholine species from natural sources (egg or soybean phosphatidylcholine) give much more permeable and less stable bilayers, whereas the saturated phospholipids with long acyl chains (for example, dipalmitoylphos phatidylcholine) form a rigid, rather impermeable bilayer structure. Liposomes useful herein can be prepared using techniques known in the art. See, Akbarzadeh et al, Nanoscale Res Lett. February 2013; 8 (1):102, which is incorporated herein by reference. In one embodiment, the liposome comprises cholesterol. It has been observed that the amount of cholesterol in the liposome composition can affect the delivery of the liposome. Thus, the amount of cholesterol may be varied. In one embodiment the amount of cholesterol in the liposome is about 10 to 50% by lipid film composition. In one embodiment, the cholesterol content of the liposome is at about 25% (moles cholesterol/total moles of lipid). In one embodiment, the cholesterol content of the liposome is about 40% (moles cholesterol/total moles of lipid). In one embodiment, the cholesterol content of the liposome is at least 25% (moles cholesterol/total moles of lipid). In one embodiment, the cholesterol content of the liposome is at least 40% (moles cholesterol/total moles of lipid). See FIG. 29 and FIG. 47.

In another aspect, the nanoparticle employed herein is a lipid nanoparticle (“LNP”). LNPs useful herein are known in the art. As used herein, LNPs are comprised of cholesterol (aids in stability and promotes membrane fusion), a phospholipid (which provides structure to the LNP bilayer and also may aid in endosomal escape), a polyethylene glycol (PEG) derivative (which reduces LNP aggregation and “shields” the LNP from non-specific endocytosis by immune cells), and an ionizable lipid (complexes negatively charged RNA and enhances endosomal escape), which form the LNP-forming composition. Fenton et al, Bioinspired Alkenyl Amino Alcohol Ionizable Lipid Materials for Highly Potent in vivo mRNA Delivery, Adv Mater. 2016 Apr. 20; 28 (15): 2939-2943, which is incorporated herein by reference.

The various components of the LNP-forming composition may be selected based on the desired target, cargo, size, etc. For example, previous studies have shown that polymeric nanoparticles made of low molecular weight polyamines and lipids can deliver nucleic acids to endothelial cells with high efficiency. Dahlman, et al, In vivo endothelial siRNA delivery using polymeric nanoparticles with low molecular weight, Nat Nanotechnol. 2014 August; 9 (8): 648-655, which is incorporated herein by reference in its entirety.

The LNP-forming composition includes an ionizable lipid or lipid-like material. As exemplified herein, in one embodiment, the ionizable lipid is C12-200. In another embodiment, the ionizable lipid is CKK-E12. In another embodiment, the ionizable lipid is 5A2-SC8. In another embodiment, the ionizable lipid is BAMEA-016B. In another embodiment, the ionizable lipid is 306O₁₀. In another embodiment, the ionizable lipid is 7C1. See, Love et al, Lipid-like materials for low-dose, in vivo gene silencing, Proceedings of the National Academy of Sciences February 2010, 107 (5) 1864-1869; Dong et al, Lipopeptides and selective siRNA delivery, Proceedings of the National Academy of Sciences March 2014, 111 (11) 3955-3960; Cheng et al, Dendrimer-Based Lipid Nanoparticles Deliver Therapeutic FAH mRNA to Normalize Liver Function and Extend Survival in a Mouse Model of Hepatorenal Tyrosinemia Type I, Advanced Materials, 30 (52) (December 2018); Liu et al, Fast and Efficient CRISPR/Cas9 Genome Editing In Vivo Enabled by Bioreducible Lipid and Messenger RNA Nanoparticles, Advanced Materials, 31 (33), August 2019; and Hajj et al, Branched-Tail Lipid Nanoparticles Potently Deliver mRNA In Vivo due to Enhanced Ionization at Endosomal pH, Small, 15 (6) (February 2019), each of which are incorporated herein by reference. Other ionizable lipids are known in the art and are useful herein.

The LNP-forming composition includes phospholipid. As exemplified herein, in one embodiment, the phospholipid (helper) is DOPE. In another embodiment, the phospholipid is DSPC. In another embodiment, the phospholipid is DOTAP. In another embodiment, the phospholipid is DOTMA. See, Kauffman et al, Optimization of Lipid Nanoparticle Formulations for mRNA Delivery in Vivo with Fractional Factorial and Definitive Screening Designs, Nano Lett., October 2015, 15 (11):7300-7306; Blakney et al, Inside out: optimization of lipid nanoparticle formulations for exterior complexation and in vivo delivery of saRNA, Gene Therapy, July 2019; and Patel et al, Lipid nanoparticles for delivery of messenger RNA to the back of the eye, J Controlled Release, 303:91-100, (June 2019), each of which are incorporated herein by reference. Other phospholipids are known in the art and are useful herein. As described by Kauffman et al, cited above, incorporation of DOPE is desirable for LNP formulations carrying mRNA.

The LNP-forming composition includes a PEG derivative. As exemplified herein, in one embodiment, the PEG derivative is a lipid-anchored PEG. In one embodiment, the lipid-anchored PEG is C14-PEG2000. In another embodiment, the lipid-anchored PEG is C14-PEG1000. In another embodiment, the lipid-anchored PEG is C14-PEG3000. In another embodiment, the lipid-anchored PEG is C14-PEG5000. In another embodiment, the lipid-anchored PEG is C12-PEG1000. In another embodiment, the lipid-anchored PEG is C12-PEG2000. In another embodiment, the lipid-anchored PEG is C12-PEG3000. In another embodiment, the lipid-anchored PEG is C12-PEG5000. In another embodiment, the lipid-anchored PEG is C16-PEG1000. In another embodiment, the lipid-anchored PEG is C16-PEG2000. In another embodiment, the lipid-anchored PEG is C16-PEG3000. In another embodiment, the lipid-anchored PEG is C16-PEG5000. In another embodiment, the lipid-anchored PEG is C18-PEG1000. In another embodiment, the lipid-anchored PEG is C18-PEG2000. In another embodiment, the lipid-anchored PEG is C18-PEG3000. In another embodiment, the lipid-anchored PEG is C18-PEG5000.

In another aspect, the nanoparticle is a protein-based nanoparticle. Nanoparticles derived from natural proteins are biodegradable, metabolizable, and are easily amenable to surface modifications to allow attachment of drugs and targeting ligands. They have been successfully synthesized from various proteins including water-soluble proteins (e.g., bovine and human serum albumin) and insoluble proteins (e.g., zein and gliadin). In one embodiment, the protein-based nanoparticle is an albumin based nanoparticle. In another embodiment, the nanoparticle is a lysozyme based nanoparticle. In yet another embodiment, the nanoparticle is a GFP based nanoparticle.

The nanoparticle is associated with a tag which comprises dibenzocyclooctyne (DBCO) covalently attached to a protein. Such tag is sometimes called “D20” or “D2ODBCO”. The dibenzocyclooctyne group (DBCO) allows copper-free click chemistry to be done with live cells, whole organisms, and non-living samples. DBCO groups will preferentially and spontaneously label molecules containing azide groups (—N3). As shown herein, in one embodiment, it is desirable to have at least 5 DBCO molecules present per molecule of protein. In one embodiment, at least 6 DBCO molecules are present per molecule of protein. In one embodiment, at least 7 DBCO molecules are present per molecule of protein. In one embodiment, at least 8 DBCO molecules are present per molecule of protein. In one embodiment, at least 9 DBCO molecules are present per molecule of protein. In one embodiment, at least 10 DBCO molecules are present per molecule of protein. In one embodiment, at least 11 DBCO molecules are present per molecule of protein. In one embodiment, at least 12 DBCO molecules are present per molecule of protein. In one embodiment, at least 13 DBCO molecules are present per molecule of protein. In one embodiment, at least 14 DBCO molecules are present per molecule of protein. In one embodiment, at least 15 DBCO molecules are present per molecule of protein. In one embodiment, at least 16 DBCO molecules are present per molecule of protein. In one embodiment, at least 17 DBCO molecules are present per molecule of protein. In one embodiment, at least 18 DBCO molecules are present per molecule of protein. In one embodiment, at least 19 DBCO molecules are present per molecule of protein. In one embodiment, at least 20 or more DBCO molecules are present per molecule of protein. In one embodiment, at least 25 or more DBCO molecules are present per molecule of protein. In one embodiment, at least 30 or more DBCO molecules are present per molecule of protein. In one embodiment, at least 35 or more DBCO molecules are present per molecule of protein. In one embodiment, at least 40 or more DBCO molecules are present per molecule of protein.

In one embodiment, the D20 tagged-nanoparticles have a diameter of about 130+/−10 nm, a PDI of less than about 0.2, or both.

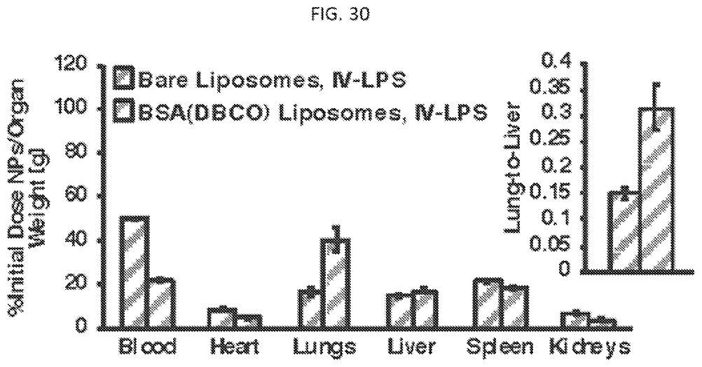

The protein used to tag the nanoparticle is desirably one that does not induce an intolerable adverse reaction, such as an immunological reaction, to the nanoparticle composition in a mammalian subject. Among suitable biomolecules are proteins that are substantially immunologically inert to mammalian subjects, particularly humans, are human albumin, bovine serum albumin, and antibodies. In one embodiment, the protein is IgG (which may be derived from any source). In another embodiment, the protein is albumin (FIG. 30).

In some embodiments, the selected D20 tagged-nanoparticles for use in this invention are loaded with one or more selected drugs. In one embodiment the selected NP contains a single drug component. In another embodiment, the selected NP is loaded with multiple drug components. By “drug” as used herein is meant any therapeutic, prophylactic or diagnostic compound or reagent that is contained within the flexible nanoparticles described herein. In one embodiment, the drug is a water-miscible compound. In one embodiment, the drug is a drug used for treating ARDS. Such drugs are known in the art, and include, without limitation, ARBs (angiotensin receptor blockers; e.g., losartan), aspirin, beta-adrenergic agonists (e.g., salmeterol, albuterol, formoterol), corticosteroids (e.g., dexamethasone, hydrocortisone, methylprednisilone), dexmedetomidine, GSK205, imatinib, inhaled nitric oxide, ketoconazole, LTRAs (leukotriene receptor antagonists; e.g., zileuton), macrolides (azithromycin, etc), methylnaltrexone, MJ33, N-acetylcysteine, NSAIDs (ibuprofen and related), pentoxifylline, roflumilast, ropivacaine, S1P-receptor agonists (fingolimod, etc)), sivelestat, SSRIs (fluoxetine, etc), statins (simvastatin, etc), thiazolidinedione (rosiglitazone, etc), vitamin C, and vitamin D. In one embodiment, multiple drugs are employed in the nanoparticles. In another embodiment, the drug is one used for treating sepsis. Drugs for treating sepsis include, without limitation, vancomycin, ceftriaxone, meropenem, ceftazidime, cefotaxime, cefepime, piperacillin, taxobactam, ampicillin, sulbactam, imipenem, levofloxacin, and clindamycin. In another embodiment, the drug is one use for treating pneumonia. Drugs for treating pneumonia include, without limitation, macrolide antibiotics (e.g., azithromycin and clarithromycin), fluoroquinolones (ciprofloxacin and levofloxacin), tetracyclines, and beta-lactams (amoxicillin, clavulanate), carbapenems, penicillins, and sulfonamides. Still other useful drugs are known in the art.

In still another embodiment, the drug is an imaging agent. Among suitable imaging agents are molecules containing radionuclides that are amenable to SPECT or PET imaging (e.g., Indium-111 for SPECT imaging); molecules containing moieties that provide contrast for CT imaging (e.g., gold nanoparticles or iodinated contrast agents); molecules containing moieties that provide contrast for MRI imaging (e.g., gadolinium); nano- or micro-scale complexes that provide contrast for ultrasound imaging (e.g., microbubbles filled with gas).

In some embodiments, the selected D20-nanoparticles for use in this invention are loaded with mRNA that encode one or more prophylactically- or therapeutically-active proteins, polypeptides, or other factors. For example, the mRNA may encode an agent that enhances tumor killing activity (such as TRAIL or tumor necrosis factor (TNF)) in a cancer. As additional non-limiting example, the mRNA may encode an agent suitable for the treatment of conditions such as muscular dystrophy (a suitable mRNAs encodes Dystrophin), cardiovascular disease (suitable mRNAs encode, e.g., SERCA2a, GATA4, Tbx5, Mef2C, Hand2, Myocd, etc.), neurodegenerative disease (suitable mRNAs encode, e.g., NGF, BDNF, GDNF, NT-3, etc.), chronic pain (suitable mRNAs encode GlyRa1, an enkephalin, or a glutamate decarboxylase (e.g., GAD65, GAD67, or another isoform), lung disease (e.g., CFTR), hemophilia (suitable mRNAs encode, e.g., Factor VIII or Factor IX), neoplasia (suitable mRNAs encode, e.g., PTEN; ATM; ATR; EGFR; ERBB2; ERBB3; ERBB4; Notch1; Notch2; Notch3; Notch4; AKT; AKT2; AKT3; HIF; H1Fla; HIF3a; Met; HRG; Bcl2; PPARalpha; PPAR gamma; WT1 (Wilms Tumor); FGF Receptor Family members (5 members: 1, 2, 3, 4, 5); CDKN2a; APC; RB (retinoblastoma); VHL; BRCA1; BRCA2; AR (Androgen Receptor); TSG101; IGF; IGF Receptor; IgfI (4 variants); Igf2 (3 variants); IgfI Receptor; Igf2 Receptor; Bax; Bcl2; caspases family (9 members: 1, 2, 3, 4, 6, 7, 8, 9, 12); Kras; Apc), age-related macular degeneration (suitable mRNAs encode, e.g., Aber; Cc12; Cc2; cp (ceruloplasmin); Timp3; cathepsinD; Vldlr), schizophrenia (suitable mRNAs encode, e.g. Neuregulinl (NrgI); Erb4 (receptor for Neuregulin); ComplexinI (CplxI); TphI Tryptophan hydroxylase; Tph2 Tryptophan hydroxylase 2; Neurexin 1; GSK3; GSK3a; GSK3b; 5-HIT (Slc6a4); COMT; DRD (DrdIa); SLC6A3; DAOA; DTNBPI; Dao (Dao1)), trinucleotide repeat disorders (suitable mRNAs encode, e.g., HTT (Huntington's Dx); SBMA/SMAXI/AR (Kennedy's Dx); FXN/X25 (Friedrich's Ataxia); ATX3 (Machado-Joseph's Dx); ATXNI and ATXN2 (spinocerebellar ataxias); DMPK (myotonic dystrophy); Atrophin-1 and Atn1 (DRPLA Dx); CBP (Creb-BP-global instability); VLDLR (Alzheimer's); Atxn7; Atxn10), fragile X syndrome (suitable mRNAs encode, e.g., FMR2; FXRI; FXR2; mGLUR5), secretase related disorders (suitable mRNAs encode, e.g., APH-1 (alpha and beta); Presenilin (Psen1); nicastrin (Ncstn); PEN-2), ALS (suitable mRNAs encode, e.g., SOD1; ALS2; STEX; FUS; TARD BP; VEGF (VEGF-a; VEGF-b; VEGF-c)), autism (suitable mRNAs encode, e.g. Mecp2; BZRAP1; MDGA2; Sema5A; Neurexin 1; Fragile X (suitable mRNAs encode, e.g., FMR2; AFF2; FXR1; FXR2; Mglur5), Alzheimer's disease (suitable mRNAs encode, e.g., E1; CHIP; UCH; UBB; Tau; LRP; PICALM; Clusterin; PS1; SORL1; CR1; Vld1r; Uba1; Uba3; CHIP28 (Aqp1, Aquaporin 1); Uchl1; Uchl3; APP), inflammation (suitable mRNAs encode, e.g., IL-10; IL-1 (IL-Ia; IL-Ib); IL-13; IL-17 (IL-17a (CTLA8); IL-17b; IL-17c; IL-17d; IL-171); 11-23; Cx3crl; ptpn22; TNFa; NOD2/CARD15 for IBD; IL-6; IL-12 (IL-12a; IL-12b); CTLA4; Cx3cll, Parkinson's Disease (suitable mRNAs encode, e.g., x-Synucicin; DJ-1; LRRK2; Parkin; PINK1), blood and coagulation disorders, such as, e.g., anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, and thalassemia (suitable mRNAs encode, e.g., CRAN1, CDA1, RPS19, DBA, PKLR, PK1, NT5C3, UMPH1, PSNI, RHAG, RH50A, NRAMP2, SPTB, ALAS2, ANH1, ASB, ABCB7, ABC7, ASAT, TAPBP, TPSN, TAP2, ABCB3, PSF2, RING11, MHC2TA, C2TA, RFXS, RFXAP, RFXS, TBXA2R, P2RX1, P2X1, HF1, CFH, HUS, MCFD2, FANCA, FACA, FA1, FA, FAA, FAAP95, FAAP90, F1134064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1), B-cell non-Hodgkin lymphoma or leukemia (suitable mRNAs encode, e.g., BCL7A, BCL7, ALI, TCLS, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AML1, WHSC1LI, NSD3, FLT3, AF1Q, NPM1, NUMA1, ZNF145, PLZF, PML, MYL, STATSB, AF1Q, CALM, CLTH, ARL11, ARLTS1, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN), inflammation and immune related diseases and disorders (suitable mRNAs encode, e.g., KIR3DL1, NKAT3, NKB1, AMB11, K1R3DS1, IFNG, CXCL12, TNFRSF6, APT1, FAS, CD95, ALPS1A, IL2RG, SCIDX1, SCIDX, IMD4, CCLS, SCYAS, D17S136E, TCP228, IL10, CSIF, CMKBR2, CCR2, CMKBRS, CCCKRS (CCRS), CD3E, CD3G, AICDA, AID, HIGM2, TNFRSFS, CD40, UNG, DGU, HIGM4, TNFSFS, CD40LG, HIGM1, IGM, FOXP3, IPEX, AIID, XPID, PIDX, TNFRSF14B, TACI), inflammation (suitable mRNAs encode, e.g., IL-10, IL-1 (IL-Ia, IL-Ib), IL-13, IL-17 (IL-17a (CTLA8), IL-17b, IL-17c, IL-17d, IL-171), 11-23, Cx3crI, ptpn22, TNFa, NOD2/CARD15 for IBD, IL-6, IL-12 (IL-12a, IL-12b), CTLA4, Cx3cII); JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, IMD4), metabolic, liver, kidney and protein diseases and disorders (suitable mRNAs encode, e.g., TTR, PALB, APOA1, APP, AAA, CVAP, AD1, GSN, FGA, LYZ, TTR, PALB, KRT18, KRT8, CIRH1A, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, SCOD1, SCO1, CTNNB1, PDGFRL, PDGRL, PRLTS, AX1NI, AXIN, CTNNB1, TP53, P53, LFS1, IGF2R, MPRI, MET, CASP8, MCHS, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, SEC63), muscular/skeletal diseases and disorders (suitable mRNAs encode, e.g., DMD, BMD, MYF6, LMNA, LMN1, EMD2, FPLD, CMDIA, HGPS, LGMDIB, LMNA, LMNI, EMD2, FPLD, CMD1A, FSHMD1A, FSHD1A, FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD1C, SEPN1, SELN, RSMD1, PLEC1, PLTN, EBS1, LRPS, BMND1, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1), neurological and neuronal diseases and disorders (suitable mRNAs encode, e.g., SOD1, ALS2, STEX, FUS, TARDBP, VEGF (VEGF-a, VEGF-b, VEGF-c), APP, AAA, CVAP, AD1, APOE, AD2, PSEN2, AD4, STM2, APBB2, FE65LI, NOS3, PLAU, URK, ACE, DCPI, ACEI, MPO, PAC1PI, PAXIPIL, PTIP, A2M, BLMH, BMH, PSEN1, AD3, Mecp2, BZRAP1, MDGA2, SemaSA, Neurexin 1, GLO1, MECP2, RTT, PPMX, MRX16, MRX79, NLGN3, NLGN4, KIAA1260, AUTSX2, FMR2, FXR1, FXR2, mGLURS, HD, IT15, PRNP, PRIP, JPH3, JP3, HDL2, TBP, SCA17, NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARKS, SNCA, NACP, PARK1, PARK4, PRKN, PARK2, PDJ, DBH, NDUFV2, MECP2, RTT, PPMX, MRX16, MRX79, CDKLS, STK9, MECP2, RTT, PPMX, MRX16, MRX79, x-Synuclein, DJ-1, Neuregulin1 (Nrg1), Erb4 (receptor for Neuregulin), Complexin1 (Cp1x1), Tph1 Tryptophan hydroxylase, Tph2, Tryptophan hydroxylase 2, Neurexin 1, GSK3, GSK3a, GSK3b, 5-HTT (Slc6a4), CONT, DRD (Drd1a), SLC6Aβ, DADA, DTNBP1, Dao (Dao1), APH-1 (alpha and beta), Presenilin (Psen1), nicastrin, (Ncstn), PEN-2, Nos1, Parp1, Nat1, Nat2, HTT, SBMA/SMAX1/AR, FXN/X25, ATX3, TXN, ATXN2, DMPK, Atrophin-1, Atn1, CBP, VLDLR, Atxn7, and Atxn10), and ocular diseases and disorders (suitable mRNAs encode, e.g., Aber, Ccl2, Cc2, cp (ceruloplasmin), Timp3, cathepsinD, Vldlr, Ccr2, CRYAA, CRYA1, CRYBB2, CRYB2, PITX3, BFSP2, CP49, CP47, CRYAA, CRYAI, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, KRIT1, APOA1, TGFBI, CSD2, CDGG1, CSD, BIGH3, CDG2, TACSTD2, TROP2, M1SI, VSX1, RINX, PPCD, PPD, KTCN, COL8A2, FECD, PPCD2, PIP5K3, CFD, KERA, CNA2, MYOC, TIGR, GLCIA, JOAG, GPOA, OPTN, GLC1E, FIP2, HYPL, NRP, CYP1BI, GLC3A, OPA1, NTG, NPG, CYP1BI, GLC3A, CRB1, RP12, CRX, CORD2, CRD, RPGRIPI, LCA6, CORDS, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, and VMD2).

In certain embodiments, the mRNA encodes a factor that can affect the differentiation of a cell. For example, expression of one or more of Oct4, Klf4, Sox2, c-Myc, L-Myc, dominant-negative p53, Nanog, Glis1, Lin28, TFIID, mir-302/367, or other miRNAs can cause the cell to become an induced pluripotent stem (iPS) cell. See also, Takahashi and Yamanaka, Cell, 126: 663-676 (2006); Takahashi, Cell, 131: 861-872 (2007); Wernig, Nature, 448: 318-324 (2007); and Yu, Science, 318: 1917-1920 (2007), the disclosures of which are incorporated herein by reference. Alternatively, the mRNA may encode a factor for transdifferentiating cells (e.g., one or more of GATA4, Tbx5, Mef2C, Myocd, Hand2, SRF, Mesp1, SMARCD3 (for cardiomyocytes); Ascl1, Nurr1, Lmx1A, Brn2, Mytl1, NeuroD1, FoxA2 (for neural cells), Hnf4α, Foxa1, Foxa2 or Foxa3 (for hepatic cells).

In another embodiment, the D20-tagged nanoparticle is further tagged or radiolabeled to allow for localization or imaging of the particles in the patient to which they've been administered. For example, the D20-tagged nanoparticles may be radiolabeled with the clinically-approved isotope indium-111. Other radioisotopes that may be used include Technetium-99m (technetium-99m), Iodine-123 and 131, Thallium-201, Gallium-67, and Fluorine-18 fluorodeoxyglucose.

The selected nanoparticle may be loaded with a suitable drug or multiple drugs generally by incubation at about 37° C. in a buffer. Desirable buffers are those that are physiologocally-compatible, such as phosphate buffered saline or the like. Other methods for drug loading include osmotic loading of a variety of small molecule drugs in the nanoparticles, allowing burst release of loaded drugs in targeted vascular beds. Drug loading and release from nanogels on RBCs may be modified by using crosslinkers incorporated in the nanogel to prolong or enhance encapsulation of loaded drugs and performing the crosslinking after drug loading. Crosslinkers can include responsive moieties (e.g. enzyme-cleavable crosslinkers that allow stimulated drug release in response to protease activity). In another embodiment, the drug is kept in the solution or in the wash buffer during all drug loading steps (except the last resuspension).

In one embodiment, the D20-tagged nanoparticle for use in the compositions described herein has no cell-specific targeting moiety or tissue-specific targeting moiety or organ-specific targeting moiety associated therewith. In another embodiment, the composition containing the D20-tagged nanoparticle has associated targeting moieties directing the composition to the target organ or tissue, such as antibodies that bind to the organ's endothelium (e.g., antibodies targeting endothelial proteins including PECAM, ICAM, VCAM, transferrin receptor, and many more) or antibodies that bind to other targeted cells. Other useful antibodies include those targeted to leukocytes (e.g., anti-CD45, -Ly6G, etc); to platelets; or to clots (e.g., antibodies binding to fibrin). In one embodiment, when an antibody is used as the protein comprised in the D20 tag, the antibody used for targeting is different. In another embodiment, the antibody used in the D20 tag and for targeting target the same cell type.

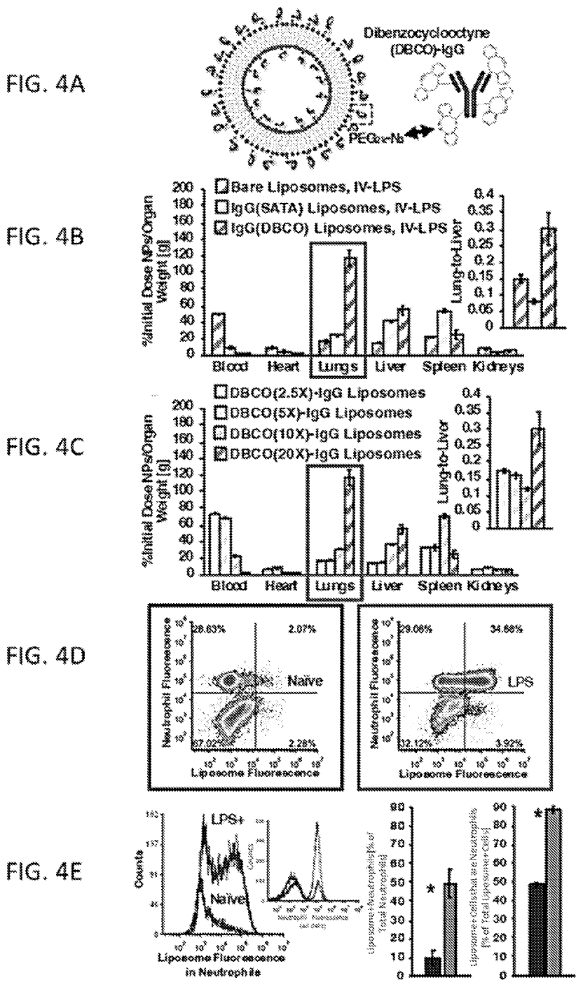

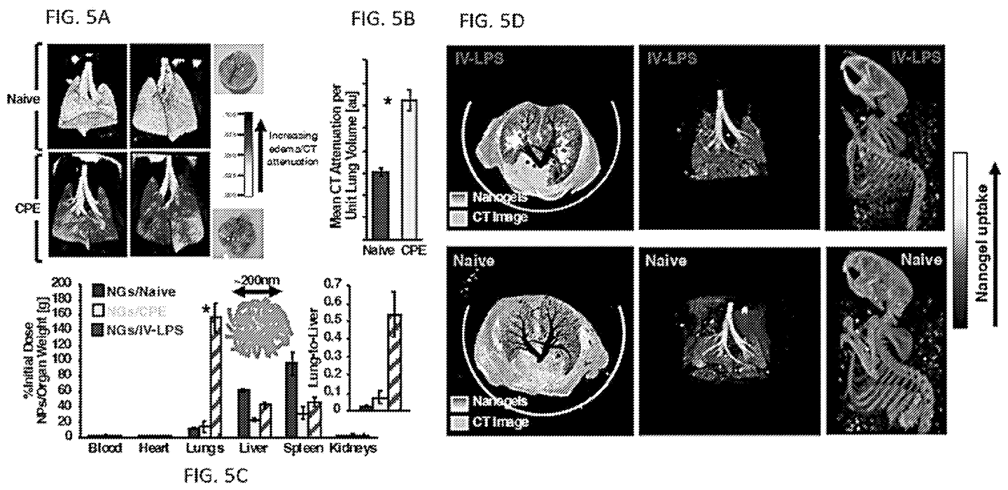

Also provided herein in another aspect is a method of generating a D20-tagged nanoparticle. In one embodiment, the selected nanoparticle (e.g., liposome) is functionalized using NHS-ester conjugation of an excess of strained alkyne (dibenzocyclooctyne, DBCO) to the selected protein (e.g., IgG), followed by reaction of the DBCO-functionalized IgG with liposomes containing PEG-azide-terminated lipids (DBCO-IgG liposomes, FIG. 4A). Various DBCO to protein ratios can be utilized, with higher ratios resulting in higher levels of DBCO present on the protein molecule. As noted herein, higher levels of DBCO on the protein molecule are desirable for targeting neutrophils. In one embodiment, the DBCO is reacted with the protein at at least a 5:1 molar ratio. In another embodiment, the DBCO is reacted with the protein at at least an 8:1 molar ratio. In another embodiment, the DBCO is reacted with the protein at at least a 10:1 molar ratio. In another embodiment, the DBCO is reacted with the protein at at least an 12:1 molar ratio. In another embodiment, the DBCO is reacted with the protein at at least a 15:1 molar ratio. In another embodiment, the DBCO is reacted with the protein at at least an 17:1 molar ratio. In another embodiment, the DBCO is reacted with the protein at at least an 18:1 molar ratio. In another embodiment, the DBCO is reacted with the protein at at least a 20:1 molar ratio. The D20 tag is covalently conjugated to the nanoparticle.

In certain embodiments, the method of generating a nanoparticle includes contacting the nanoparticle with serum or serum proteins (e.g., diluted in a suspension with nanoparticles). In certain embodiments, the method of generating a nanoparticle includes contacting the nanoparticle with one or more complement proteins (e.g. diluted in a suspension with nanoparticles). Complement proteins include, e.g., C1, C4, C2, C3, C5, C6, C7, C8, and C9, and fragments thereof, including cleavage products (e.g., C4 is cleaved to C4b) (see Ling, M., & Murali, M. (2019). Analysis of the Complement System in the Clinical Immunology Laboratory. Clin Lab Med. 2019 December; 39 (4):579-590, which is incorporated herein by reference). In certain embodiments, following an incubation period the nanoparticles are isolated from the suspension or undergo washing to remove serum or complement proteins and/or to concentrate the nanoparticles in a solution. In certain embodiments, the method of generating a nanoparticle includes a dialysis step and/or centrifugation to wash and/or concentrate the nanoparticles in a solution.

In another aspect, provided herein are methods of treating lung injury in a subject in need thereof. The method includes administering D20-tagged nanoparticles to a subject in need thereof. By “subject” is meant primarily a human, but also domestic animals, e.g., dogs, cats; and livestock, such as cattle, pigs, etc.; common laboratory mammals, such as primates, rabbits, and rodents; and pest or wild animals, such as deer, rodents, rabbits, squirrels, etc. In one embodiment, the nanoparticles are used to treat pneumonia. In another embodiment, the nanoparticles are used to treat sepsis.

In certain embodiments, the nanoparticles are used to treat acute respiratory distress syndrome (ARDS). ARDS is an acute, diffuse, inflammatory lung injury with a variety of causes, most commonly pneumonia and sepsis. ARDS causes the lungs' air sacs, called alveoli, to fill up with proteinaceous liquid, preventing the lungs from oxygenating the blood. The impact of ARDS is enormous, with 190,000 US cases per year, and a mortality rate of 35%. Decades of research have yielded myriad drug targets, but after the failure of more than a dozen large clinical trials, there are still no FDA approved drugs that improve survival in ARDS. From a pharmacology perspective, there are three reasons why many rationally chosen drugs have failed in ARDS. Firstly, ARDS patients are too fragile to tolerate drug side effects. These patients have multi-organ dysfunction, and thus cannot tolerate even mild side effects. Secondly, the inhalational route of delivery, useful for so many pulmonary problems, has limited benefit in ARDS, as the flooded alveoli (those filled with liquid) are covered by a column of fluid, which means that topical delivery to the alveoli is not possible via the inhaled route. Finally, ARDS is a very heterogeneous disease, so targeting a single pathway is unlikely to be sufficient.

In another aspect, provided herein are methods of treating inflammation or treating an inflamed tissue. The method includes administering D20-tagged nanoparticles to a subject in need thereof. The inflammation may be attributable, e.g., to an inflammatory disorder and/or infection in the subject. In certain embodiments, method of treating subacute or acute inflammation in an inflame tissue are provided.

As demonstrated herein, when the D20 tag is conjugated onto translatable nanocarriers such as liposomes, the liposomes accumulate in inflamed, but not naïve, lungs at ˜20% of the injected dose (% ID), which is a level of targeting similar to the previous best tag for driving liposomes into the lungs, anti-PECAM antibodies. Notably, the lungs contain the majority of the body's marginated neutrophils in sepsis, ARDS, and pneumonia. Further, the D20-tagged liposomes described herein are effective to decrease ARDS phenotype, even without drugs loaded in the nanoparticles. Thus, in one embodiment, D20-tagged nanoparticles are administered to a patient having, or suspected of having ARDS.

In another embodiment, it is desirable to incorporate drugs that will concentrate in the affected microvasculature into the D20-tagged nanoparticles. For example, for sepsis, D20-tagged liposomes are loaded with drugs that limit neutrophil damage (inhibitors of neutrophil elastase, NETosis, etc) all of which exist but barely reach neutrophils before being cleared using current therapies. Thus, in another embodiment, an ARDS-treating drug is loaded into the nanoparticles prior to administration to the patient. In another embodiment, a sepsis-treating drug is loaded into the nanoparticles prior to administration to the patient. In another embodiment, a pneumonia-treating drug is loaded into the nanoparticles prior to administration to the patient. Such drugs are known in the art, and include, without limitation, ARBs (angiotensin receptor blockers; e.g., losartan), aspirin, beta-adrenergic agonists (e.g., salmeterol, albuterol, formoterol), corticosteroids (e.g., dexamethasone, hydrocortisone, methylprednisilone), dexmedetomidine, GSK205, imatinib, inhaled nitric oxide, ketoconazole, LTRAs (leukotriene receptor antagonists; e.g., zileuton), macrolides (azithromycin, etc), methylnaltrexone, MJ33, N-acetylcysteine, NSAIDs (ibuprofen and related), pentoxifylline, roflumilast, ropivacaine, S1P-receptor agonists (fingolimod, etc), sivelestat, SSRIs (fluoxetine, etc), statins (simvastatin, etc), thiazolidinedione (rosiglitazone, etc), vitamin C, and vitamin D.

In one embodiment of the method, vascular permeability is decreased as compared to a control as a result of the treatment. In another embodiment, protein leakage in the alveoli is decreased as a result of the treatment. In yet another embodiment, cellular infiltration in the alveoli is decreased as a result of the treatment.

In another aspect, a method of targeting leukocytes is provided. The method includes administering the D20-tagged nanoparticles as described herein. The desired target of the nanoparticles can be any leukocyte, including neutrophils, monocytes, macrophages, eosinophils, basophils, NK cells, lymphocytes, dendritic cells. In one embodiment, the leukocytes are marginated leukocytes. The delivery of nanoparticles with tropism for neutrophils can result in enhanced uptake of the nanoparticles by neutrophils, which then leave the lung vasculature or an inflamed tissue. In this manner, injury or inflammation can be alleviate by decreasing the number and/or concentration of neutrophils. In certain embodiments, a method of targeting and depleting neutrophils in the lung vasculature is provided. In certain embodiments, a method of targeting and depleting neutrophils in an inflamed tissue is provided.

In certain embodiments, vascular permeability in an inflamed tissue is reduced as compared to a control as a result of treatment. In certain embodiments, protein leakage in the inflamed tissue is reduced as a result of treatment. In certain embodiments, cellular infiltration in the inflamed tissue is reduced as a result of treatment. In certain embodiments, the concentration and/or number of neutrophils in the inflamed tissue and/or tissue vasculature is reduced as a result of treatment.

In another aspect, a method of diagnosing a condition associated with lung injury is provided. The condition associated with lung injury is, in one embodiment, on that results in, or has as a symptom of, marginated leukocytes. Marginated leukocytes are those white blood cells that accumulate inside the vasculature of affected organs, directly in contact with the inner wall of the blood vessels (predominantly capillaries) (See, e.g., Hogg, J. C. Physiol. Rev. 67, 1249-1295 (1987); Doerschuk, C. M. et al. J. Appl. Physiol. 63, 1806-1815 (1987); and Kuebler, W. M. & Goetz, A. E. Eur. Surg. Res. 34, 92-100 (2002), which are incorporated herein by reference). Marginated leukocytes (especially marginated neutrophils) massively increase their numbers during sepsis, ARDS, and pneumonia, and have a major role in these diseases (See, e.g., Brown, K. A. et al. Lancet 368, 157-169 (2006) and Stiel, L., Meziani, F. & Helms, J. Shock 49, 371-384 (2018), which are incorporated herein by reference). The marginated neutrophils clog the microvasculature, release toxic mediators such as proteases and reactive oxygen species, produce pro-inflammatory cytokines, and induce clotting and further inflammation by releasing neutrophil extracellular traps (NETs) (See, e.g., Brown, K. A. et al. Lancet 368, 157-169 (2006) and Lelubre, C. & Vincent, J.-L. Nat. Rev. Nephrol. 14, 417-427 (2018), which are incorporated herein by reference). All of these marginated leukocyte functions lead to organ dysfunction. Thus, technologies to identify the presence of marginated neutrophils and modulate their activity serve as major new diagnostics and therapeutics for sepsis, ARDS, and pneumonia.

In certain embodiments, radiolabeled D20-tagged liposomes are administered to a patient suspected of having a condition associated with marginated leukocytes. In one embodiment, the radiolabel is the clinically-approved isotope indium-111. If an increase of indium-111 signal in the chest (due to abundant marginated neutrophils in the lungs in the disease of interest) is observed, the subject has a disease associated with marginated leukocytes, such as ARDS, sepsis, or pneumonia. In one embodiment, the subject is then treated for said condition using one or more of the drugs mentioned herein or known in the art. In another embodiment, additional testing is performed, to aid in the diagnosis. For example, in one embodiment, a chest X-ray is performed to determine whether there is water-density material in the airspaces. By correlating the X-ray opacities with the presence of increased D20-In-111 signal, a diagnosis of a disease associated with marginated leukocytes, such as ARDS, sepsis, or pneumonia is made.

Administration of the nanoparticle compositions described herein can be intravenously for delivery of the drug to the lungs. For example, where the disease is ARDS, pneumonia, or sepsis, the compositions may be administered intravenously. For administration to any other organ or tissue, the composition may be administered intravenously or intra-arterially immediately upstream of an organ for delivery of effective doses of the drug. Other routes of administration useful herein include intra-arterial; e.g., delivery via intra-arterial catheter in the middle cerebral artery immediately after endovascular thrombectomy for an ischemic stroke, where neutrophils are common; topical delivery to a wound (e.g., after surgery); intra-articular (into the joint space, e.g., during a flare of autoimmune and inflammatory arthritis); into any infectious or inflammatory fluid-filled space (intra-pleural, intra-peritoneal, intra-thecal; intra-ocular; intra-ventricular/cisterna); into an abscess.

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

The terms “a” or “an” refers to one or more, for example, “a nanoparticle” is understood to represent one or more nanoparticles. As such, the terms “a” (or “an”), “one or more,” and “at least one” are used interchangeably herein.

The term “about” as used herein when referring to a measurable value such as an amount, a temporal duration, and the like, is meant to encompass variations of up to ±10% from the specified value; as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.”

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

### EXAMPLES

The following examples disclose specific embodiments of preparation of compositions of this invention, their characteristics, and uses. These examples should be construed to encompass any and all variations that become evident as a result of the teachings provided.

Nanoparticle structural properties that shape interactions with neutrophils in the setting of acute inflammation are described herein. Due to the key role of neutrophils in lung physiology and the pathology of acute lung disease (noted above for its broad clinical impact) and the high concentration of neutrophils in the lung vasculature, we focused on the localization of nanoparticles to the lung vasculature in LPS injury models. After verifying the intravascular presence of activated neutrophils in mouse lungs subjected to LPS-induced inflammation, we showed that two nanoparticles, lysozyme-dextran nanogels and crosslinked albumin nanoparticles, selectively home to neutrophils in inflamed lungs, but not naïve lungs.

In tracing the biodistributions of 23 different nanoparticles in naïve mice and in mice subjected to model sepsis, we observed that a diverse range of protein-based nanostructures avidly localize to acutely inflamed lungs, but not naïve lungs. We showed that 13 amorphous protein nanostructures, defined by hydrophobic protein interactions, NHS-ester protein crosslinking, and association of charged proteins, have specificity for LPS-injured lungs. Conversely, we demonstrated that three crystalline protein nanostructures, adenovirus, adeno-associated virus, and ferritin, have biodistributions unaffected by LPS injury. We also showed that polystyrene nanoparticles and five liposome formulations do not have specificity for injured lungs, indicating that nanostructures not based on protein are not intrinsically drawn to pulmonary neutrophils in acute inflammation. Finally, we demonstrated that liposomes can be engineered for affinity to neutrophils in LPS-injured lungs by coating with IgG densely modified with a hydrophobic cyclooctyne tag.

Towards demonstrating translational applicability of this structure-based targeting mechanism, we applied our neutrophil-specific nanoparticles in diagnostic imaging experiments, characterization of therapeutic effects in model ARDS, and targeting to ex vivo human lungs rejected for donation due to injury. Specifically, we showed a) that lysozyme-dextran nanogels have capacity for diagnostic imaging contrast (SPECT-CT) that distinguishes acute inflammatory lung injury from cardiogenic edema; b) that liposomes modified for neutrophil affinity can ameliorate the neutrophil-mediated effects of model ARDS; and c) that lysozyme-dextran nanogels, but not ferritin nanocages, have affinity for leukocytes resident in excised human lungs rejected for transplant due to injury.

Taken together, our results show that a broad range of protein-based nanostructures may comprise agents with intrinsic ability to target neutrophils in acute inflammation. Our imaging data, therapeutic results in model ARDS, and targeting results in injured human lungs and inflamed tissues indicate that protein-based nanoparticles with a broad range of amorphous structures have therapeutic and diagnostic applications as agents that specifically target acute inflammation without the need for affinity tags.

### Example 1

### Characterization of Neutrophil Content and Function in Inflamed Lungs

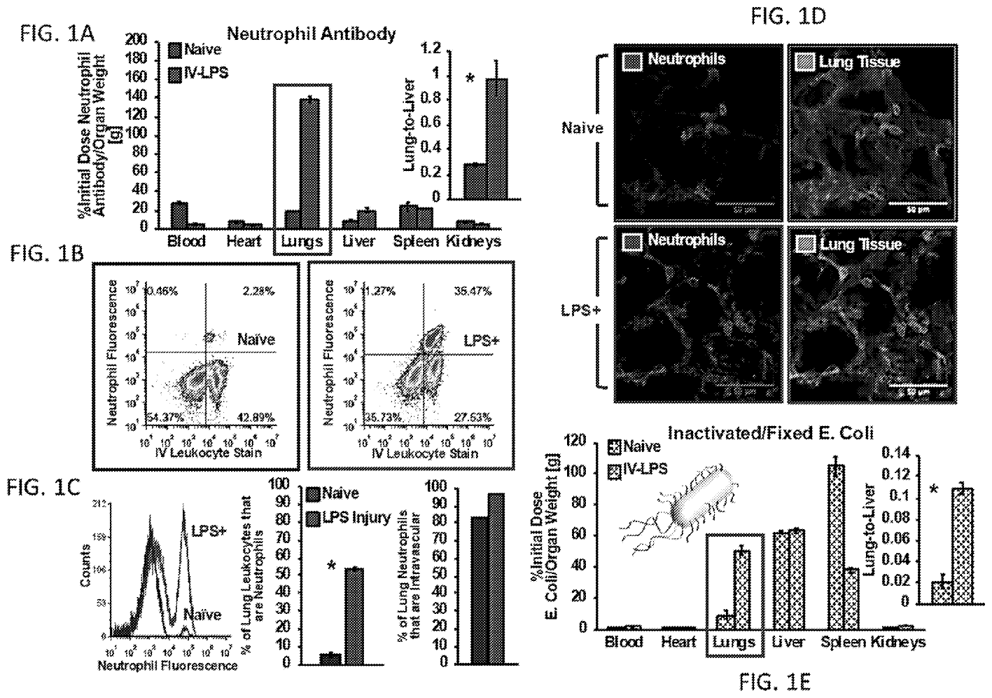

Radiolabeled clone 1A8 anti-Ly6G antibody was administered to determine the location and concentration of neutrophils in naïve mice and mice exposed to intravenous (IV) lipopolysaccharides (LPS) (FIG. 1A). Accumulation of anti-Ly6G antibody in the lungs was dramatically affected by LPS injury, with 20.81% of injected antibody adhering in LPS-injured lungs, compared to 2.82% of injected antibody in naïve control lungs. Antibody circulation time was also reduced by systemic LPS injury. Agreeing with previous studies addressing the role of neutrophils in systemic inflammation, biodistributions of anti-Ly6G antibody indicated that systemic LPS injury profoundly increased the concentration of neutrophils in the lungs and moderately increased the concentration of neutrophils in the liver.

Single cell suspensions prepared from mouse lungs were probed in flow cytometry to further characterize pulmonary neutrophils in naïve mice and in mice following LPS-induced systemic inflammation. In order to identify intravascular populations of leukocytes, mice received intravenous fluorescent CD45 antibody five minutes prior to sacrifice. Single cell suspensions prepared from IV CD45-stained lungs were then stained with anti-Ly6G antibody to identify neutrophils. A second stain of single cell suspensions with CD45 antibody indicated the total population of leukocytes in the lungs, distinct from the intravascular population indicated by IV CD45.

Flow cytometry showed greater concentrations of neutrophils in LPS-injured lungs, compared to naïve lungs (FIG. 1B, counts above horizontal threshold indicate positive staining for neutrophils, FIG. 1C, rightmost peak indicates positive staining for neutrophils). Comparison of Ly6G stain to total CD45-positive cells indicated 53.53% of leukocytes in the lungs were neutrophils after LPS injury, compared to 5.62% in the naïve control (FIG. 1C, center panel). Comparison of Ly6G stain to IV CD45 stain indicated that the majority of neutrophils were intravascular, in both naïve and LPS-injured mice. In naïve mice, 83.04% of neutrophils were intravascular and in LPS-injured mice, 96.60% of neutrophils were intravascular (FIG. 1C, right panel). Large populations of intravascular neutrophils following inflammatory injury is consistent with previously published observations.

Histological analysis confirmed results obtained with flow cytometry and radiolabeled anti-Ly6G biodistributions. Namely, staining of lung sections indicated increased concentration of neutrophils in the lungs following IV LPS injury (FIG. 1D, left panels). Co-registration of neutrophil staining with autofluorescence (indicating tissue architecture) broadly supported the finding that pulmonary neutrophils reside in the vascular space of the lungs (FIG. 1D, right panels).

Previous work has traced the neutrophil response to bacteria in the lungs, determining that pulmonary neutrophils pursue and engulf active bacteria following either intravenous infection or infection of the airspace in the lungs. We injected heat-inactivated, oxidized, and fixed E. coli in naïve and IV-LPS-injured mice. With the bacteria stripped of their functional behavior by heat treatment, oxidation, and fixation, E. coli did not accumulate in the lungs of naïve control mice (1.47% of initial dose in the lungs, FIG. 1E). However, pre-treatment with LPS to recapitulate the inflammatory response to infection led to enhanced accumulation of the deactivated E. coli in the lungs (7.69% of initial dose in the lungs, FIG. 1E). With E. coli structure maintained but E. coli function removed, the inactivated bacteria were taken up in pulmonary neutrophils primed by an inflammatory injury.

### Injury-Specific Uptake of Nanoparticles in Pulmonary Neutrophils

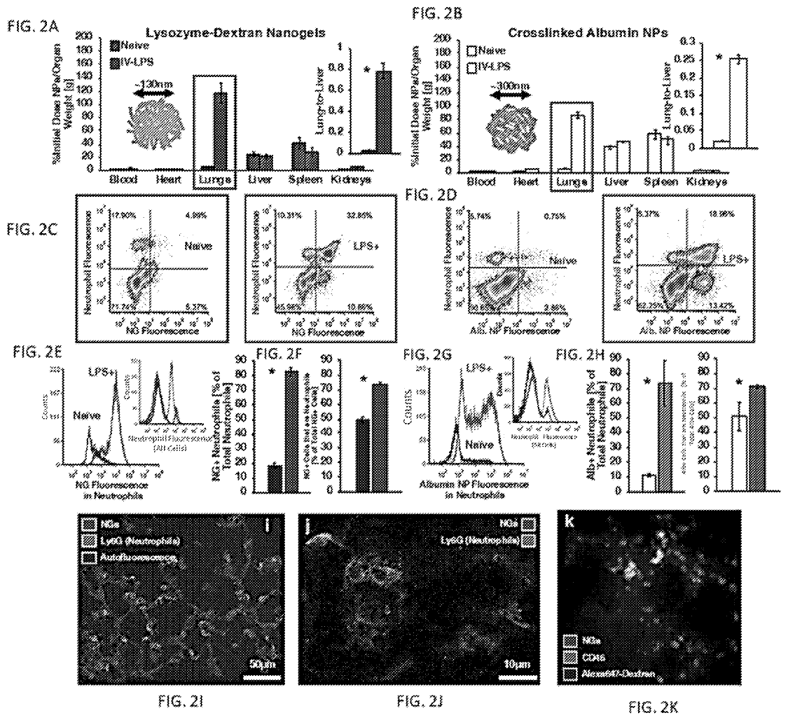

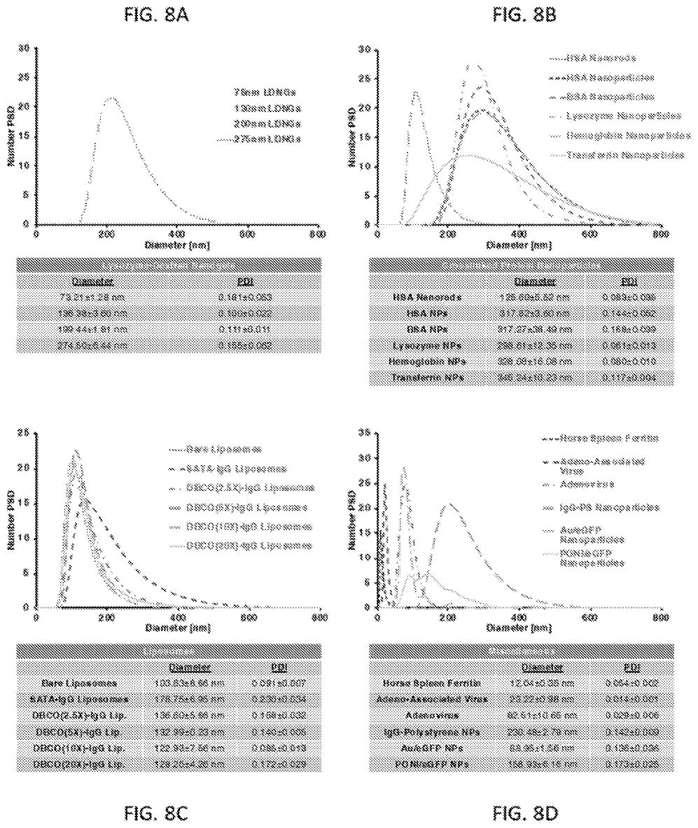

Lysozyme-dextran nanogels (LDNGs, NGs) and poly(ethylene)glycol (PEG)-crosslinked albumin nanoparticles have been characterized as targeted drug delivery agents in previous work. Here, LDNGs (136.38±3.60 nm diameter, 0.100±0.022 PDI, FIG. 8A) and PEG-crosslinked albumin NPs (317.82±3.60 nm diameter, 0.144±0.052 PDI, FIG. 8B) were administered in naïve and IV-LPS-injured mice. Neither nanoparticle was functionalized with antibodies or other affinity tags. The protein component of each particle was labeled with ¹²⁵I for tracing in biodistributions assessed 30 minutes after IV administration of nanoparticles. In naïve lungs, bare LDNGs accumulated at a concentration of 5.25 percent initial dose per gram organ weight (% ID/g). After IV LPS injury, LDNGs accumulated in the lungs at 116.43% ID/g. Both absolute LDNG lung uptake and ratio of lung uptake to liver uptake registered a ˜25-fold increase between naïve control and LPS-injured animals (FIG. 2A). Specificity for LPS-injured lungs was recapitulated with PEG-crosslinked human albumin NPs. Albumin NPs accumulated in naïve lungs at 5.23% ID/g, and in LPS-injured lungs at 87.62% ID/g, accounting for a 17-fold increase in lung uptake after intravenous LPS insult (FIG. 2B).

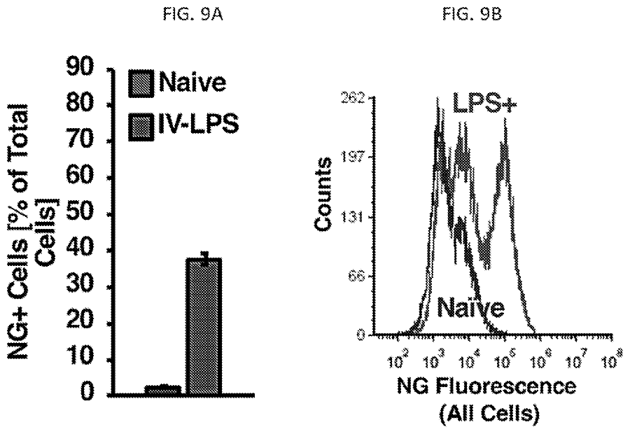

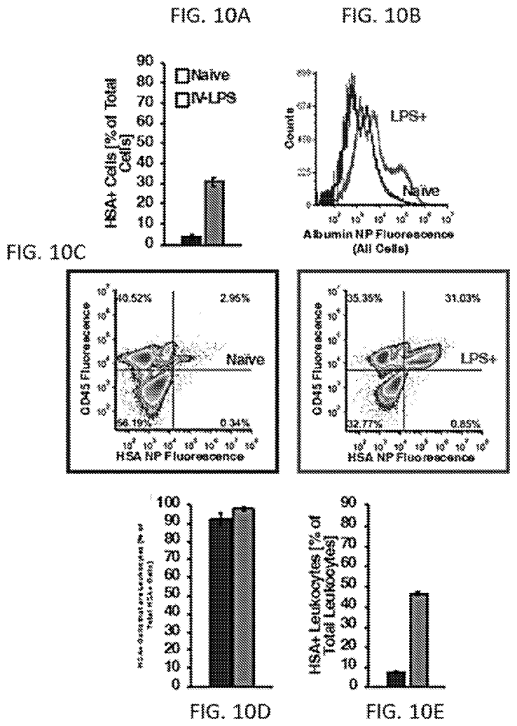

Single cell suspensions were prepared from lungs after administration of fluorescent LDNGs or PEG-crosslinked albumin NPs. Flow cytometric analysis of cells prepared from lungs after NP administration enabled identification of cell types with which NPs associated. Firstly, the total number of cells containing LDNGs or albumin NPs increased between naïve and LPS-injured lungs. In naïve control lungs, 2.23% of cells were positive for LDNGs and 4.37% of cells were positive for albumin NPs. In LPS-injured lungs, 37.62% of cells were positive for LDNGs and 31.30% of cells were positive for albumin NPs (FIG. 9A, FIG. 9B, FIG. 10A, FIG. 10B).

Ly6G stain for neutrophils indicated that the bulk of LDNG and albumin NP accumulation in LPS-injured lungs was accounted for by uptake in neutrophils. In FIG. 2C-FIG. 2D, counts above the horizontal threshold indicate neutrophils and counts to the right of the vertical threshold indicate cells containing LDNGs (FIG. 2C) or Albumin NPs (FIG. 2D). In the naïve lungs, low levels of NP uptake are distributed between neutrophils and other cells. In IV-LPS-injured lungs, more neutrophils are present and LDNG and albumin NP uptake is dominated by neutrophils (FIG. 2C, FIG. 2D, upper right quadrants indicate NP-positive neutrophils). 82.51% of neutrophils were positive for LDNGs in LPS-injured lungs, compared to 18.53% in naïve lungs (FIG. 2E, FIG. 2F). 73.71% of neutrophils were positive for albumin NPs in LPS-injured lungs, compared to 11.39% in naïve lungs (FIG. 2G, FIG. 2H). Notably, even in the naïve lungs, neutrophils played a significant role in the low levels of LDNG and albumin NP uptake. In naïve lungs, 49.19% of LDNG-positive cells and 50.62% of albumin NP-positive cells were neutrophils. By comparison, neutrophils accounted for 74.00% of LDNG-positive cells and 70.59% of albumin NP-positive cells in LPS-injured lungs (FIG. 2F, FIG. 2H, rightmost panels). For NP uptake not accounted for by neutrophils, CD45 staining indicated that the remaining NP uptake was attributable to other leukocytes. Colocalization of albumin NP fluorescence with CD45 stain showed that 91.98% of albumin NP uptake was localized to leukocytes in naïve lungs and 97.83% of albumin NP uptake was localized to leukocytes in injured lungs (FIG. 10C, FIG. 10D).

For LDNGs, localization to neutrophils in injured lungs was confirmed via histology. Ly6G staining of LPS-injured lung sections confirmed colocalization of fluorescent nanogels with neutrophils in the lung vasculature (FIG. 2I). Slices in confocal images of lung sections indicated that LDNGs were inside neutrophils (FIG. 2J). Finally, intravital images of injured lungs allowed real-time visualization of LDNG uptake in leukocytes in injured lungs. LDNG fluorescent signal accumulated over 30 minutes and reliably colocalized with CD45 staining for leukocytes (FIG. 2K).

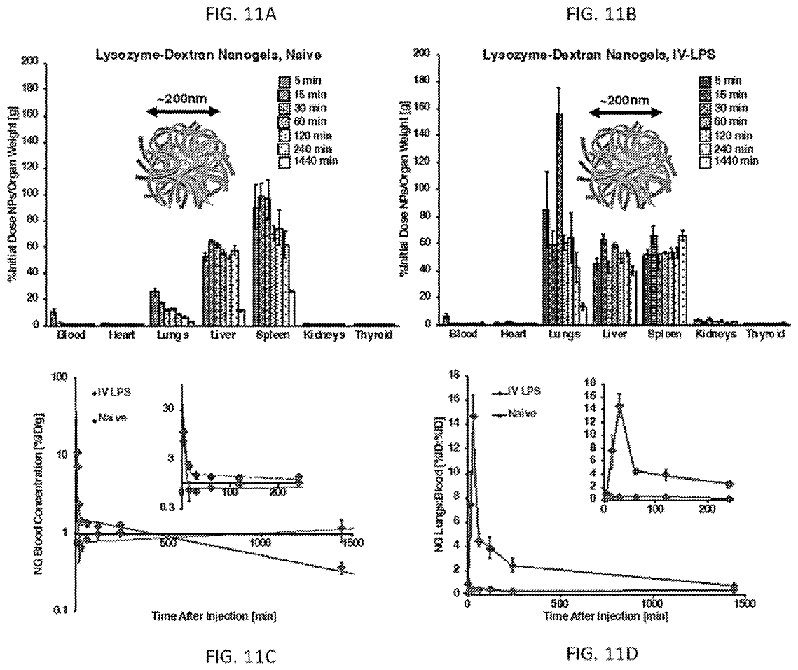

LDNG pharmacokinetics were evaluated in naïve and IV-LPS-injured mice (FIG. 11). In both naïve and injured mice, bare LDNGs were rapidly cleared from the blood with a distribution half-life of ˜3 minutes. In naïve mice, transient retention of LDNGs in the lungs (25.91% ID/g at five minutes after injection) leveled off over one hour. In IV-LPS-treated mice, LDNG concentration in the lungs reached a peak value at 30 minutes after injection, as measured either by absolute levels of lung uptake or by lungs:blood localization ratio.

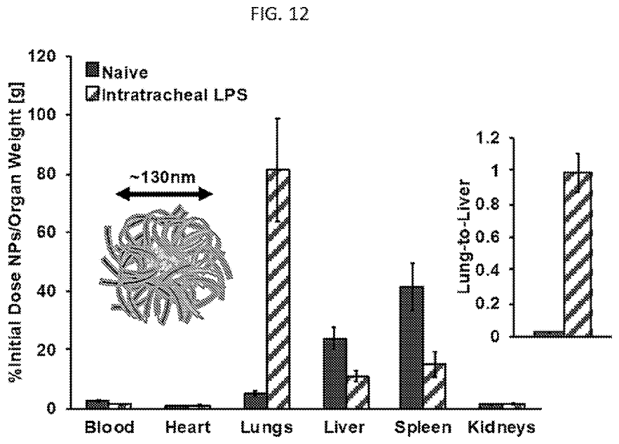

LDNG biodistributions were also assessed in mice undergoing alternative forms of LPS-induced inflammation. Intratracheal (IT) instillation of LPS led to concentration of LDNGs in the lungs at 81.31% ID/g. Liver and spleen LDNG uptake was also reduced following IT LPS injury, leading to a 45-fold increase in the lungs:liver LDNG localization ratio induced by IT LPS injury (FIG. 12). As with IV LPS injury, IT LPS administration leads to neutrophil-mediated vascular injury focused in the lungs.

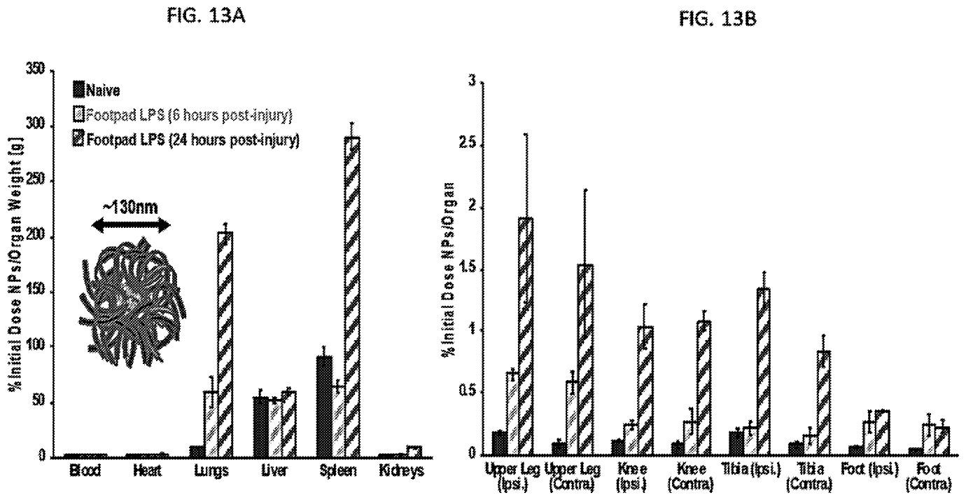

As a model of local infection spreading to systemic inflammation, mice were administered LPS via footpad injection. LDNGs uptake in the lungs and in the legs was enhanced by footpad LPS administration. At 6 hours after footpad LPS administration, LDNGs concentrated in the lungs at 59.29% ID/g, an 11-fold increase over naïve. At 24 hours, LDNGs concentrated in the lungs at 202.64% ID/g (FIG. 13A). Total LDNG accumulation in the legs accounted for 0.850 percent initial dose (% ID) in naïve mice, 2.650% ID in mice 6 hours after footpad LPS injection, and 8.343% ID at 24 hours after footpad injection (FIG. 13B).

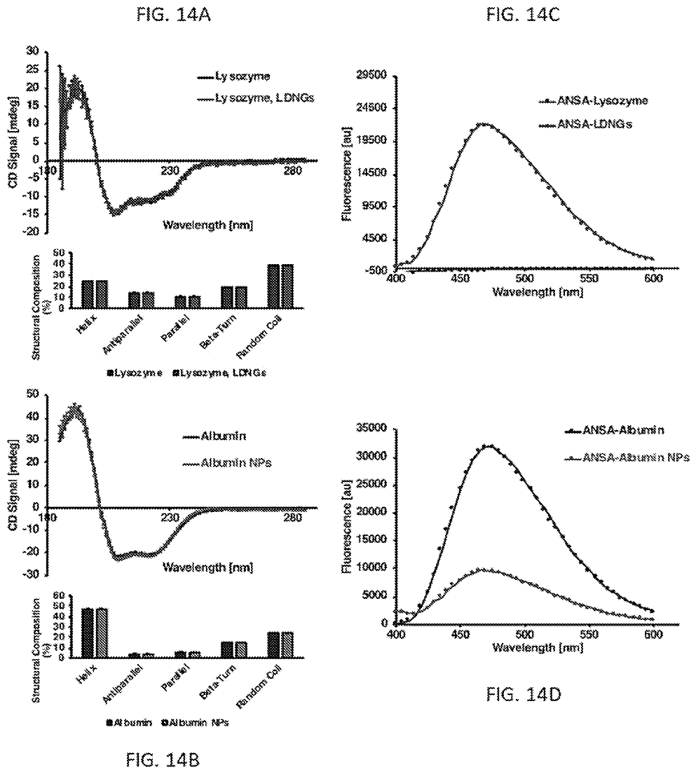

Previous work has indicated that albumin NPs generated by denaturing albumin in organic solvent accumulate in neutrophils in inflamed lungs and at the site of acute injury, where nanoparticles coated with native albumin do not. We have characterized lysozyme-dextran nanogels and crosslinked human albumin NPs with circular dichroism (CD) spectroscopy to compare secondary structure of proteins in the NPs to secondary structure of the native component proteins (FIG. 14A and FIG. 14B). Identical CD spectra were recorded for LDNGs vs. lysozyme and for albumin NPs vs. human albumin, with concentration of the free proteins set to match the quantity of protein in the NPs. Deconvolution of the CD spectra via neural network algorithm trained against a library of CD spectra for known structures verified that secondary structure of lysozyme and albumin was unchanged by incorporation of the proteins in the NPs.

Free protein and protein NPs were also probed with 8-anilino-1-naphthalenesulfonic acid (ANSA), previously demonstrated as a tool for determining the extent to which hydrophobic domains are exposed on proteins in native gels. Consistent with previous work on the structure of the two studied proteins, ANSA staining indicated little available hydrophobic domains on lysozyme and substantial hydrophobic exposure on albumin (FIG. 14C and FIG. 14D). However, LDNGs had increased hydrophobic accessibility vs. native lysozyme and albumin NPs had reduced hydrophobic accessibility vs. native albumin. Therefore, our data indicate that lysozyme and albumin are not denatured in LDNGs and albumin NPs, but accessibility of hydrophobic domains in the two proteins is altered by incorporation in the NPs.

### In Vivo Tracing of Diverse Protein Nanoparticle Structures Following Neutrophil-Mediated Acute Lung Injury

Variants of LDNG structure, crosslinked protein NP structure, and NP structure based on charged protein interactions were traced in naïve control and IV-LPS-injured mice. As examples of highly crystalline protein-based NPs based on site-specific protein interactions (rather than site-indiscriminate interactions leading to crosslinker, gelation, or charge-based protein NPs), we also traced viruses in naïve and LPS-treated mice. Adding in liposomes and polystyrene NPs as example nanostructures not based on protein, we undertook a study to evaluate how aspects of NP structure including size, composition, surface chemistry, and crystallinity impact NP affinity for LPS-injured lungs.

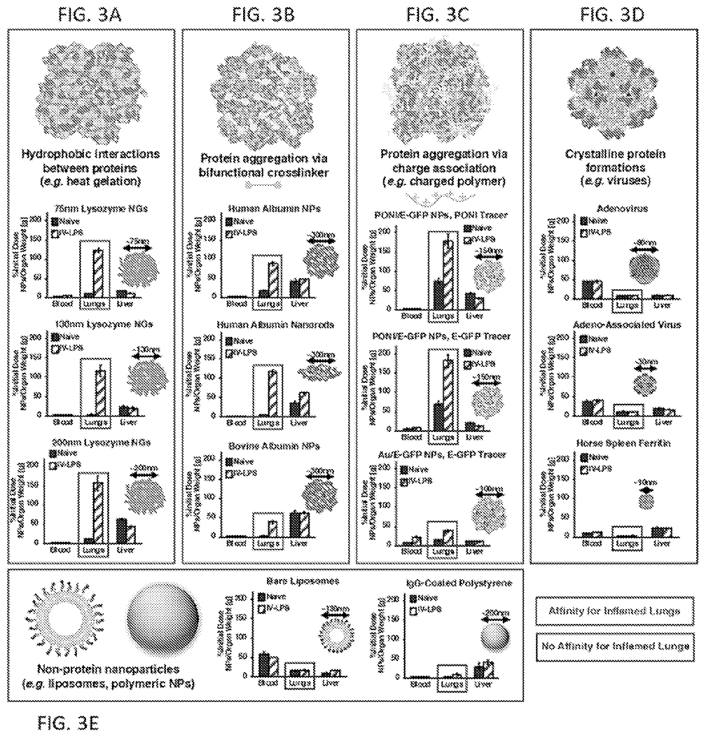

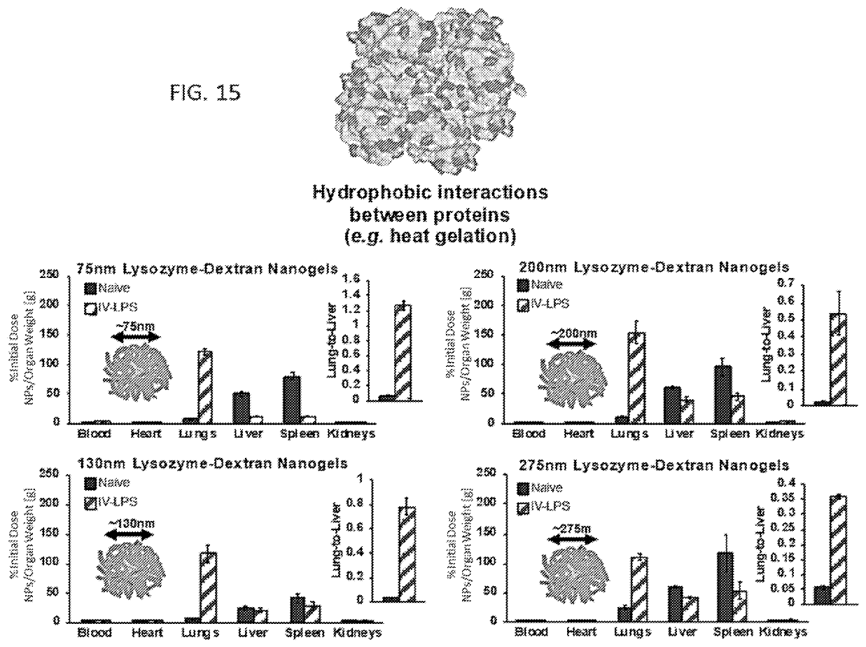

Firstly, LDNG size was varied by modifying lysozyme-dextran composition of the NPs and pH at which particles were formed. LDNGs of ˜75 nm (73.21±1.28 nm, PDI 0.181±0.053), ˜200 nm (199.44±1.81 nm, PDI 0.111±0.011), and ˜275 nm (274.50±6.44 nm, PDI 0.155±0.062) diameter were traced in naïve control and IV-LPS-injured mice, adding to data obtained for 130 nm LDNGs above (FIG. 3A, FIG. 8A, FIG. 15). As with data for 130 nm LDNGs reported in FIG. 2, all sizes of LDNGs accumulated in LPS-injured lungs with much greater avidity than in naïve lungs. 75 nm LDNGs localized to inflamed lungs at a concentration of 122.27% ID/g, compared to 7.49% ID/g in naïve lungs. 200 nm LDNGs accumulated at 156.05% ID/g in LPS-injured lungs, and at 12.17% ID/g in naïve lungs. 275 nm bare LDNGs accumulated in injured lungs at a concentration of 110.54% ID/g, compared to 22.39% ID/g in naïve lungs. Based on lung:liver localization ratio, even 275 nm LDNGs exhibited a 6.5-fold increase in lung affinity after LPS induction of inflammation. Variations in structure and composition of LDNGs therefore did not affect LDNG affinity for LPS-injured lungs.

Expanding on data with PEG-NHS ester crosslinked human serum albumin particles, we varied the geometry and protein composition of nanoparticles based on PEG-NHS crosslinked protein. Human albumin nanorods (aspect ratio 3:1), bovine albumin nanoparticles (317.27±38.49 nm, PDI 0.168±0.039), human hemoglobin nanoparticles (328.08±16.08 nm, PDI 0.080±0.010), human transferrin nanoparticles (345.24±10.23 nm, PDI 0.117±0.004), and chicken lysozyme nanoparticles (298.61±12.35 nm, PDI 0.061±0.013) were traced in naïve and IV LPS-injured mice (FIG. 3B, FIG. 8B, FIG. 11). With the exception of crosslinked lysozyme nanoparticles, all of the tested formulations had clear specificity for acutely inflamed lungs over naïve lungs. Lysozyme nanoparticles accumulated in naïve lungs at a uniquely high concentration of 137.47% ID/g, compared to 170.92% ID/g in inflamed lungs. Among other protein NPs, we record LPS:naïve lung uptake ratios of 18.88 for human albumin nanorods, 9.90 for bovine albumin NPs, 3.00 for human hemoglobin NPs, and 2.97 for human transferrin NPs. Therefore, acute inflammatory injury resulted in a minimum three-fold increase in lung uptake for all examined crosslinked protein nanoparticles, excluding crosslinked lysozyme, which still accumulated in injured lungs at 25.64% of initial dose. Nonetheless, degree of uptake in injured lungs, along with injured vs. naïve contrast, did vary with protein NP composition.

To represent a third class of protein NP, comprising protein accumulated in NPs via charge interactions, we employed recently-developed nanoparticles based on poly(glutamate) tagged green fluorescent protein (E-GFP). Negatively-charged E-GFP was paired to arginine-presenting gold nanoparticles (88.95±1.56 nm diameter, PDI 0.136±0.036) or to poly(oxanorborneneimide) (PONT) functionalized with guanidino and tyrosyl side chains (158.93±6.16 nm diameter, PDI 0.173±0.025) (FIG. 8D). For biodistribution experiments with PONI/E-GFP hybrid NPs, tyrosine-bearing PONI was labeled with ¹³¹I and E-GFP was labeled with ¹²⁵I, allowing simultaneous tracing of each component of the hybrid NPs. The two E-GFP NPs, with structure based on charge interactions, had specificity for IV LPS-injured lungs. Comparing uptake in LPS-injured lungs to naïve lungs, we observe an LPS:naïve ratio of 2.37 for PONI/E-GFP NPs as traced by the PONI component, 2.57 for PONI/E-GFP NPs as traced by the E-GFP component, and 2.79 for Au/E-GFP NPs (FIG. 3C, FIG. 12). PONI/E-GFP particles, specifically, accumulated in LPS-injured lungs at 26.77% initial dose as measured by PONI tracing (27.24% initial dose as measured by GFP tracing).

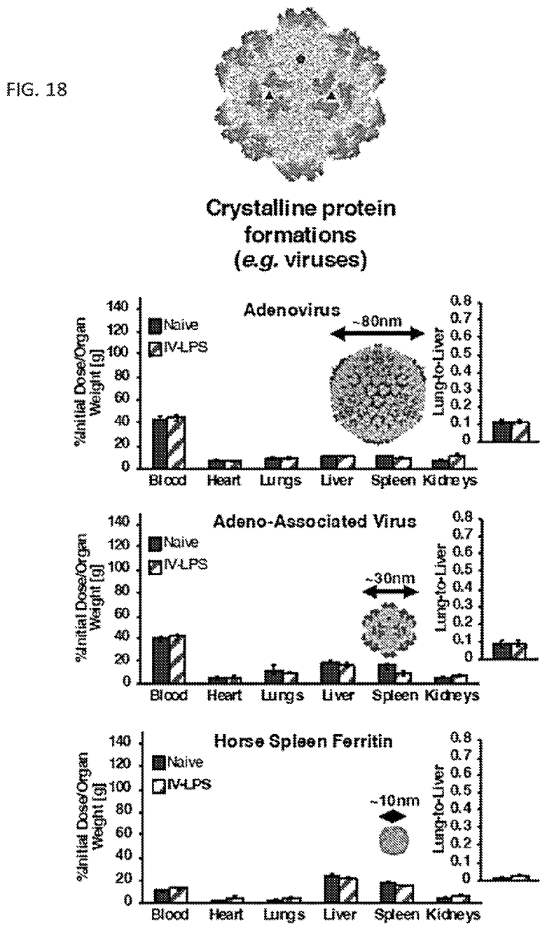

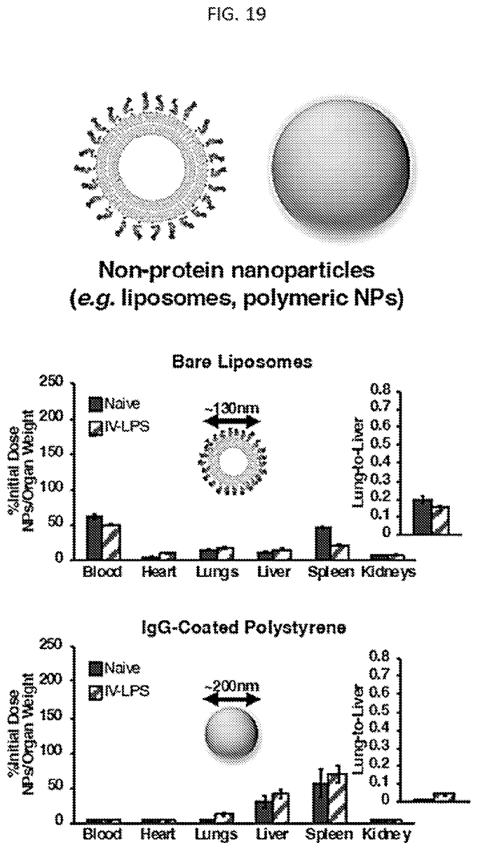

Finally, adeno-associated virus, adenovirus, and horse spleen ferritin nanocages were employed as examples of protein-based nanoparticles with structure based on symmetrical and site-specific protein interactions (FIG. 8D for confirmation of structure). For each of these crystalline protein nanoparticles, IV LPS injury had no significant effect on biodistribution (FIG. 3D, FIG. 18). LPS:naïve lung uptake ratios were 1.01 for adenovirus, 0.80 for adeno-associated virus, and 1.15 for horse spleen ferritin, with no significant differences noted in inflamed vs. naïve values for any of the particles. Adenovirus concentrated in LPS-injured lungs at 1.34% initial dose, adeno-associated virus at 1.50% initial dose, and ferritin at 0.68% initial dose. Therefore, crystalline protein nanoparticles traced in our studies did not have specificity for the lungs after acute inflammatory injury.

Liposomes and polystyrene NPs were studied as examples of nanoparticle structure not based on protein. Bare liposomes incorporated DOTA chelate-containing lipids, allowing labeling with ¹¹¹In tracer for biodistribution studies. Carboxylated polystyrene NPs were coupled to trace amounts of ¹²⁵I labeled IgG via EDCI-mediated carboxy-amine coupling. Liposomes had a diameter of 103.63±8.66 nm (PDI 0.091±0.007) and IgG-polystyrene NPs had a diameter of 230.48±2.79 nm (PDI 0.142±0.009) (FIG. 8C-8D). Neither bare liposomes nor polystyrene NPs were drawn to LPS-injured lungs in significant concentrations (FIG. 3E, FIG. 19). Liposomes accumulated in inflamed lungs at a concentration of 16.89% ID/g, accounting for no significant change against naïve lungs. LPS injury actually induced a fall in the lungs:liver metric, from 0.20 for naïve mice to 0.15 for LPS-injured mice. Polystyrene NPs accumulated in inflamed lungs at 11.67% ID/g (1.75% initial dose). Though absolute levels of lung uptake were low for IgG-coated polystyrene NPs, IV LPS injury did in fact induce increased levels of NP uptake in the lungs, from a concentration of 2.40% ID/g in the naïve lungs.

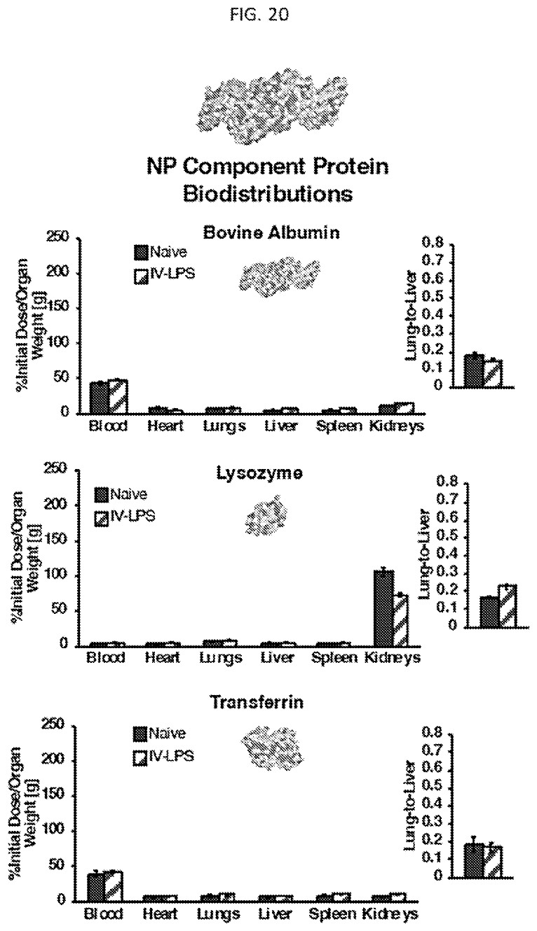

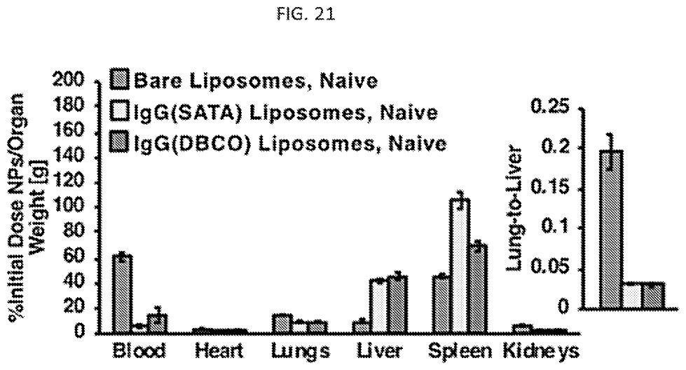

Notably, isolated proteins did not home to LPS-inflamed lungs themselves. Among protein components of the tested NPs, we traced radiolabeled bovine albumin, lysozyme, and transferrin in naïve control and IV LPS-injured mice (FIG. 20). In injured mice, bovine albumin, lysozyme, and transferrin localized to the lungs at concentrations of 9.22% ID/g (1.38% initial dose), 8.92% ID/g (1.34% initial dose), and 9.69% ID/g (1.45% initial dose), respectively. No significant differences were recorded when comparing naïve to LPS-injured lung uptake for isolated proteins.

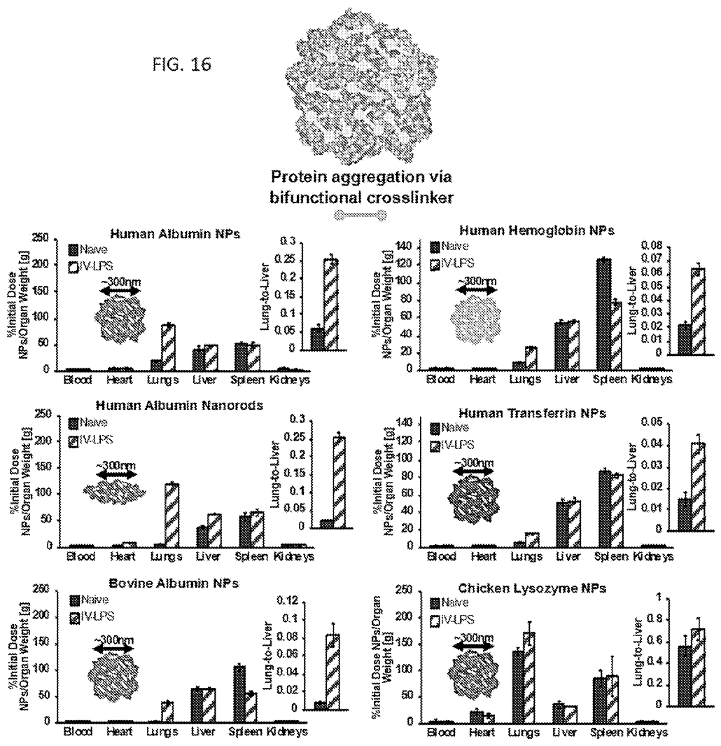

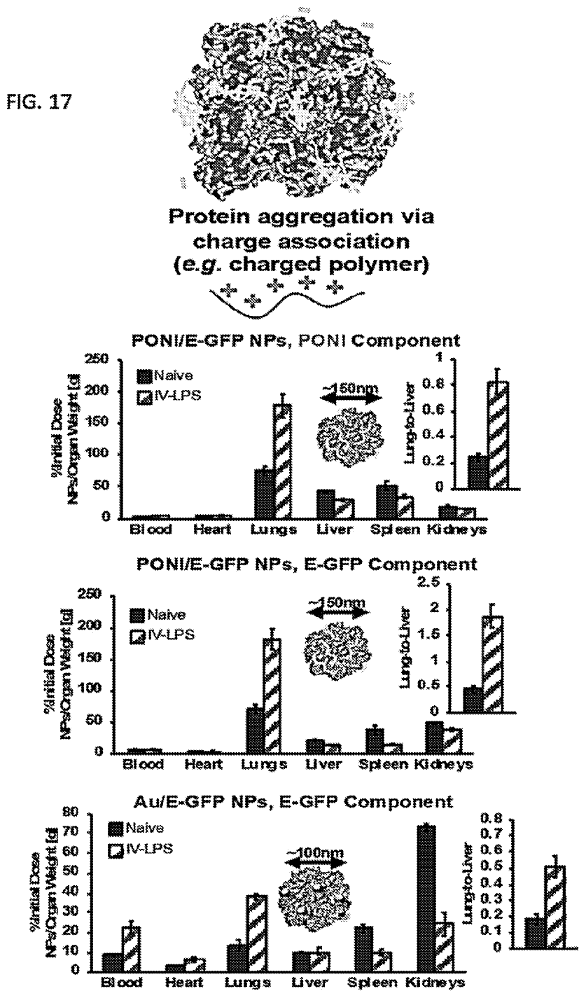

The data presented in FIG. 3A-FIG. 3E and FIG. 15-FIG. 20 indicate that a variety of protein-based nanostructures can target acute inflammatory injury in the lungs. Namely, NPs based on agglutination of proteins in non-site-specific interactions (FIG. 3A-FIG. 3C) all exhibited either significant increases in lung uptake after LPS injury or high levels of lung uptake in both naïve control and LPS-injured animals (in the case of crosslinked lysozyme NPs, FIG. 16). Highly symmetrical protein nanostructures, namely viruses and ferritin nanocages, had no affinity for inflamed lungs (FIG. 3D). As examples of nanostructures not based on protein, bare liposomes and polystyrene beads did not home to inflamed lungs.

### Engineering of Immunoliposome Surface Chemistry for Structure-Based Targeting to Neutrophils in Acutely Injured Lungs

Liposomes, traced by ¹¹¹In-labeled chelate-conjugated lipid, were functionalized with rat IgG conjugated via SATA-maleimide chemistry (SATA-IgG liposomes) or via recently demonstrated copper-free click chemistry methods. SATA-IgG liposomes had a diameter of 178.75±6.95 nm and a PDI of 0.230±0.034 (FIG. 8C). Briefly, click chemistry methods entailed NHS-ester conjugation of an excess of strained alkyne (dibenzocyclooctyne, DBCO) to IgG, followed by reaction of the DBCO-functionalized IgG with liposomes containing PEG-azide-terminated lipids (DBCO-IgG liposomes, FIG. 4a). DBCO-IgG liposomes had a diameter of 128.25±4.26 nm and a PDI of 0.172±0.029 (FIG. 8C).

In mice subjected to IV-LPS injury, SATA-IgG liposomes accumulated in the lungs at a concentration of 22.26% ID/g (FIG. 4B). DBCO-IgG liposomes, by contrast, concentrated in the lungs at 117.16% ID/g, corresponding to 17.57% of initial dose and roughly matching the accumulation of 130 nm LDNGs in the injured lungs (FIG. 4B). For comparison, bare liposomes, as in FIG. 3E, concentrated in the injured lungs at 16.89% ID/g (FIG. 4B). The three types of liposomes accumulated in naïve lungs at comparatively uniform levels of 14.75% ID/g for bare liposomes, 10.69% ID/g for SATA-IgG liposomes, and 9.86% ID/g for DBCO-IgG liposomes (FIG. 2I). For DBCO-IgG liposomes, the injured vs. naïve lung uptake accounted for a twelve-fold change.

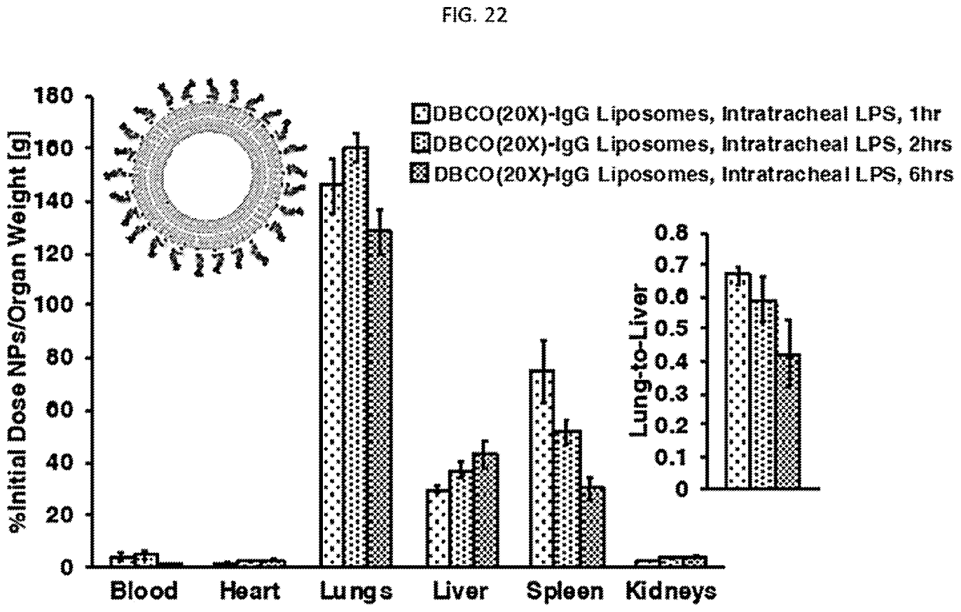

High concentrations of DBCO-IgG liposomes also accumulated in mouse lungs after IT LPS instillation. Biodistributions of the DBCO-IgG liposomes indicated a concentration of 145.89% ID/g at 1 hour after IT LPS, 160.13% ID/g at 2 hours after IT LPS, and 127.78% ID/g at 6 hours after IT LPS (FIG. 22). It is notable that, even at early time points after direct pulmonary LPS insult, DBCO-IgG liposomes accumulated in the inflamed lungs.

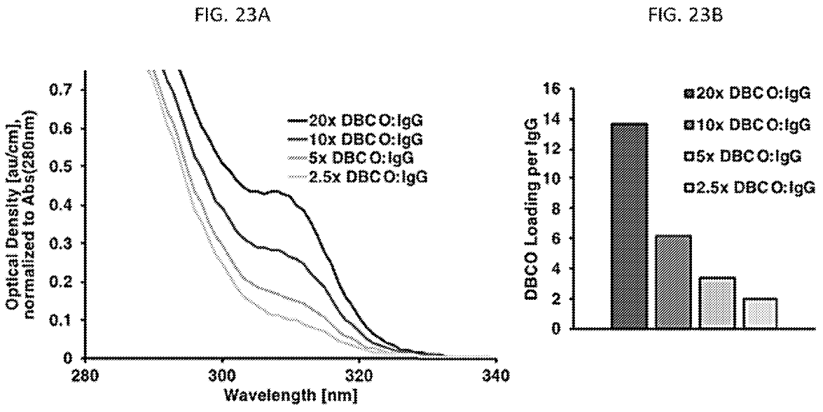

Results in FIG. 4B were obtained by introducing a 20-fold molar excess of NHS-ester-DBCO to rat IgG before DBCO-IgG conjugation to liposomes (DBCO(20×)-IgG liposomes). Optical density quantification of DBCO indicated ˜14 DBCO per IgG following reaction of DBCO and IgG at 20:1 molar ratio (FIG. 23). To test the hypothesis that DBCO functions as a tag that modifies DBCO-IgG liposomes for neutrophil affinity in settings of inflammation, we varied the concentration of DBCO on IgG prepared for conjugation to azide liposomes. DBCO was added to IgG at 10-fold, 5-fold, and 2.5-fold molar excesses. A 10-fold molar excess resulted in ˜6 DBCO per IgG, a 5-fold molar excess resulted in ˜3 DBCO per IgG, and a 2.5-fold molar excess resulted in ˜2 DBCO per IgG (FIG. 23). IgG with different DBCO loading concentrations was conjugated to azide liposomes. DBCO-IgG liposomes had similar sizes across all DBCO concentrations (FIG. 8C). Namely, all DBCO-IgG liposomes had a diameter ˜130 nm and a PDI<0.20. The different types of DBCO-IgG liposomes were each traced in IV LPS injured mice. Titrating the quantity of DBCO on DBCO-IgG liposomes indicated that targeting to the lungs of injured mice was dependent on DBCO concentration on the liposome surface (FIG. 4C). Compared to 117.16% ID/g lung uptake for DBCO(20×)-IgG liposomes, DBCO(10×)-IgG liposomes concentrated in injured lungs at 31.35% ID/g, DBCO(5×)-IgG liposomes at 17.79% ID/g, and DBCO(2.5×)-IgG liposomes at 16.91% ID/g. Therefore, only IgG with high concentrations of DBCO served as a tag for modifying the surface of liposomes for targeting to pulmonary injury.

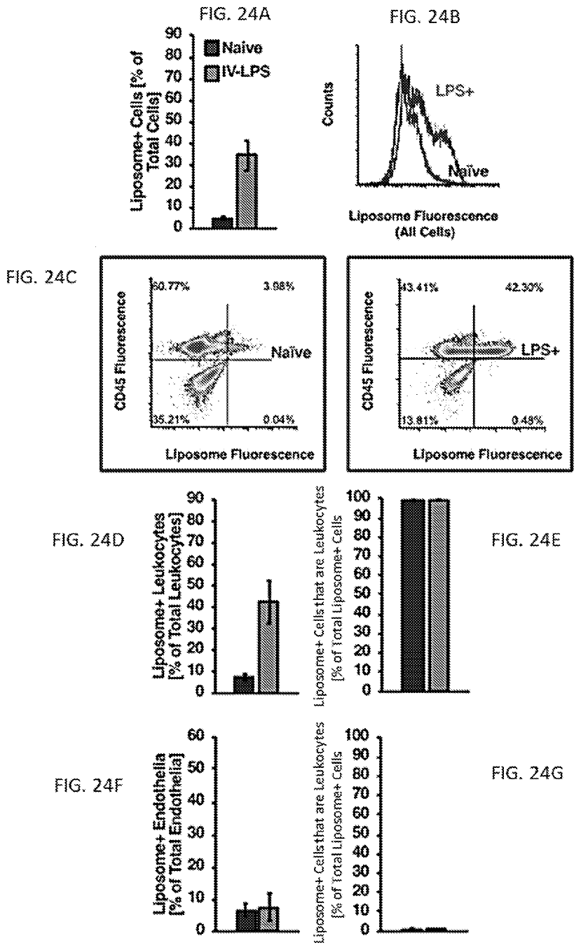

Flow cytometry verified the specificity of DBCO-IgG liposomes for neutrophils in injured lungs (FIG. 4D-FIG. 4E). As with LDNGs and albumin NPs in FIG. 2C-2H, single cell suspensions were prepared from LPS-injured and naïve control lungs after circulation of fluorescent DBCO-IgG liposomes. Confirming the results of biodistribution studies, 4.90% of cells were liposome-positive in naïve lungs, compared to 33.92% of all cells in LPS-injured lungs (FIG. 24A and FIG. 24B).

DBCO-IgG liposomes predominantly accumulated in pulmonary neutrophils after IV LPS. There were more neutrophils in the injured lungs and a greater fraction of neutrophils took up DBCO-IgG liposomes in the injured lungs, as compared to naïve control (FIG. 4D and FIG. 4E). In naïve lungs, 9.68% of neutrophils contained liposomes, compared to 49.46% in IV LPS-injured lungs. DBCO-IgG liposomes were also highly specific for neutrophils in inflamed lungs. 88.51% of liposome-positive cells were also positive for Ly6G stain in injured lungs, compared to 48.36% in the naïve lungs. The remaining DBCO-IgG liposome uptake in the lungs was accounted for by other CD45-positive cells (FIG. 24C-FIG. 24E). 99.04% of liposome uptake colocalized with CD45-positive cells in LPS-injured lungs and 98.73% of liposome uptake in the naïve lungs was associated with CD45-positive cells. Accordingly, less than 1% of liposome uptake was associated with endothelial cells (FIG. 24F and FIG. 24G).

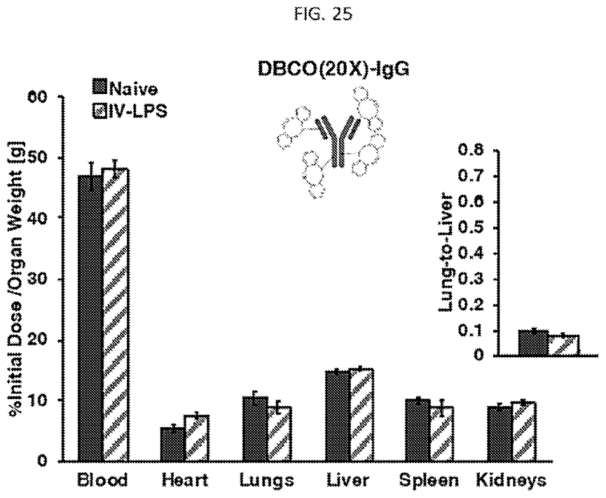

DBCO(20×)-IgG itself did not have specificity for inflamed lungs (FIG. 25). Uptake of DBCO(20×)-IgG in naïve and injured lungs was statistically identical and the biodistribution of the modified IgG resembled published results with unmodified IgG. These results verify that DBCO-IgG acts to modify the structure of immunoliposomes, but does not function as a standard affinity tag by comprising a surface chemistry with intrinsic affinity for neutrophils.

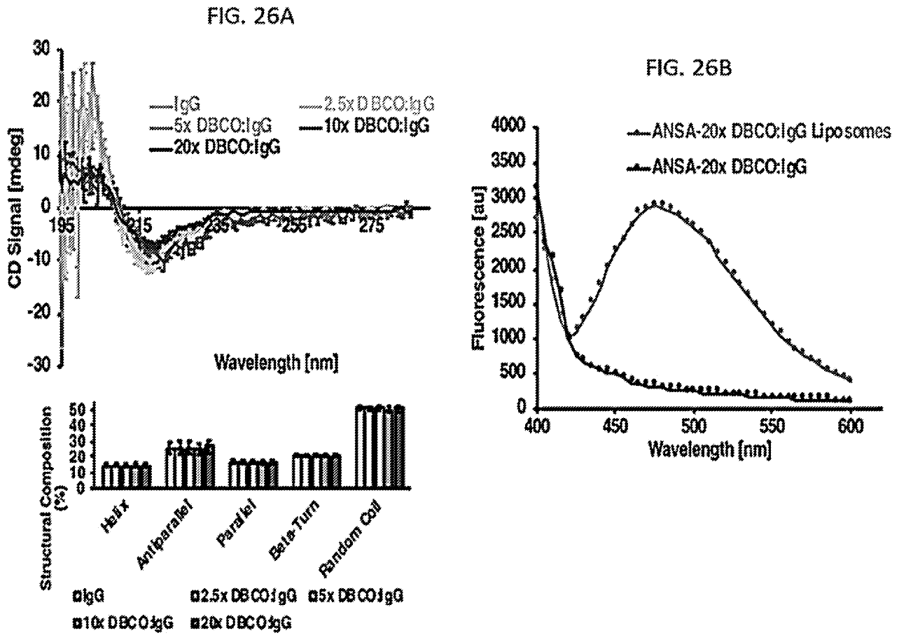

Indeed, CD spectroscopic and ANSA structural characterization of DBCO-modified IgG and DBCO-IgG liposomes resembled results obtained for LDNGs and crosslinked albumin NPs. IgG secondary structure, as assessed by CD spectroscopy, was unchanged by DBCO modification (FIG. 26A). Deconvolution of CD spectra via neural network algorithm indicated identical structural compositions for DBCO(20×)-IgG, DBCO(10×)-IgG, DBCO(5×)-IgG, DBCO(2.5×)-IgG, and unmodified IgG. ANSA was used to probe accessible hydrophobic domains on DBCO(20×)-IgG and DBCO(20×)-IgG liposomes (FIG. 26B). ANSA fluorescence indicated more hydrophobic domains available on DBCO(20×)-IgG liposomes than on DBCO(20×)-IgG itself, resembling results for lysozyme and LDNGs.

### Imaging Lung Inflammation With Neutrophil-Targeted Nanoparticles

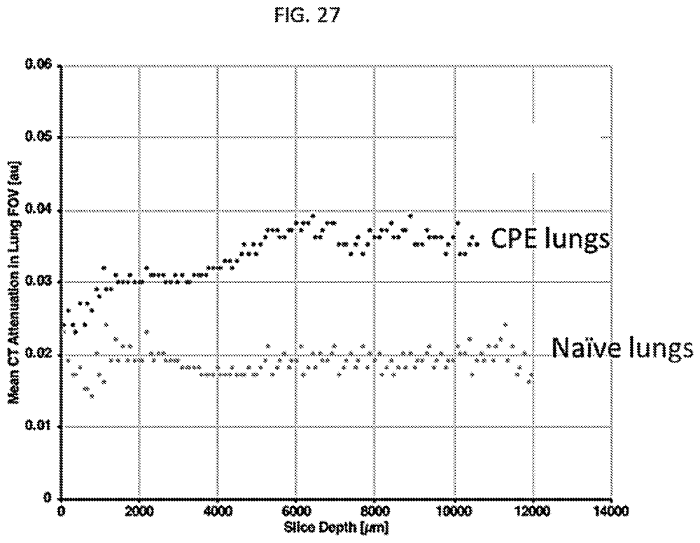

Computerized tomography (CT) imaging is a standard diagnostic tool for ARDS. CT images can identify the presence of edematous fluid in the lungs, but CT identification of edema cannot distinguish cardiogenic pulmonary edema from edema originating with vascular damage in ARDS. We implemented a mouse model of cardiogenic pulmonary edema induced via protracted propranolol infusion. Edema was confirmed via CT imaging of inflated lungs ex vivo and in situ. In FIG. 5A, three dimensional reconstructions of chest CT images were partitioned to indicate airspace and low-density tissue, as in normal lungs, with white, yellow, and light orange signal. Partitioning of CT signal also allowed high-density tissue and edema to be indicated with red and black/transparent signal. Quantification of CT attenuation and gaps in the reconstructed three-dimensional lung images indicated profuse edema in lungs afflicted with model cardiogenic pulmonary edema (FIG. 5A, FIG. 5B, FIG. 27).

200 nm LDNGs were traced in mice with induced cardiogenic pulmonary edema. LDNGs accumulated in the edematous lungs at 14.52% ID/g concentration, statistically identical to lung uptake in naïve mice and an order of magnitude lower than the level of lung uptake in mice treated with IV LPS (FIG. 5C).

Naïve and IV LPS-injured mice were dosed with LDNGs labeled with ¹¹¹In via chelate conjugation to lysozyme. ¹¹¹In uptake in naïve and LPS-injured lungs was visualized with ex vivo SPECT-CT imaging to indicate capacity of LDNGs for imaging-based diagnosis of inflammatory lung injury (FIG. 5D). ¹¹¹In signal was colocalized with anatomical CT images for reconstructions in FIG. 5D. ¹¹¹In SPECT signal was detectable in LPS-injured lungs, but ¹¹¹In SPECT signal was at background level in naïve lungs. Reduced SPECT signal in the liver of LPS-injured mice, in agreement with biodistribution data, was also evident in co-registration of SPECT imaging with full body skeletal CT imaging.

### Therapeutic Effects of Neutrophil-Targeted Nanoparticles in Model ARDS

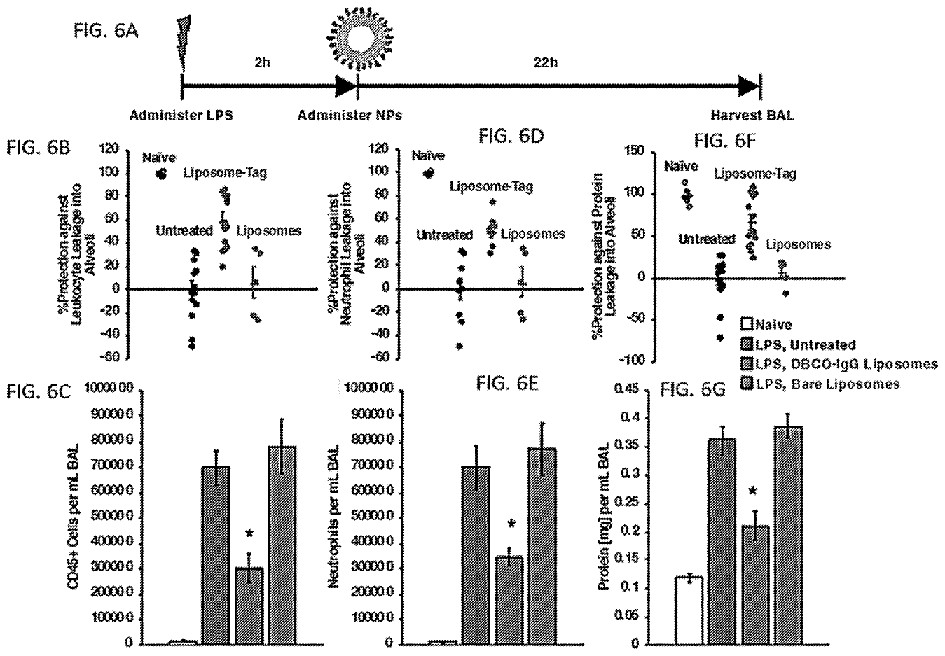

Mice were treated with nebulized LPS as a high-throughput model for ARDS. To evaluate physiological effects of the model injury, bronchoalveolar lavage (BAL) fluid was harvested from mice at 24 hours after exposure to LPS. In three separate experiments, nebulized LPS induced elevated concentrations of neutrophils, CD45-positive cells, and protein in the BAL fluid. In naïve mice, CD45-positive cells concentrated at 0.142×105 cells per mL BAL and neutrophils concentrated at 0.111×10⁵ cells per mL BAL. After LPS injury, CD45-positive cells and neutrophils concentrated at 6.968×10⁵ and 6.964×10⁵ cells per mL BAL, respectively. Vascular disruption after nebulized LPS treatment led to accumulation of protein-rich edema in the alveolar space. In naïve mice, protein concentrated in the BAL fluid at 0.119 mg/mL and in LPS-injured mice, protein concentrated in the BAL at 0.361 mg/mL (FIG. 6G).

DBCO(20×)-IgG liposomes were compared to bare liposomes for effects on vascular permeability in model ARDS. Liposomes were administered as an IV bolus (20 mg per kg body weight) two hours after nebulized LPS administration (FIG. 6A-FIG. 6G). As in untreated mice, BAL fluid was harvested and analyzed at 24 hours after exposure to nebulized LPS. Bare liposomes did not have an effect on vascular injury induced by nebulized LPS. In BAL fluid from mice receiving bare liposomes, CD45-positive cells and neutrophils concentrated at 7.817×10⁵ and 7.673×10⁵ cells per mL, respectively. Following bare liposome treatment, LPS-injured mice had 0.388 mg/mL of protein in the BAL fluid. DBCO(20×)-IgG liposomes, however, had a significant salient effect on both protein leakage and cellular infiltration in the BAL. With DBCO(20×)-IgG liposomes administered two hours after nebulized LPS, CD45-positive cells and neutrophils in BAL were reduced to concentrations of 3.041×10⁵ and 3.477×10⁵ cells per mL, respectively. Protein concentration in the BAL was reduced to 0.211 mg/mL by DBCO(20×)-IgG liposome treatment. As measured by protection against cellular or protein leakage, relative to untreated mice, DBCO(20×)-IgG liposomes provided 59.57% protection against leukocyte leakage, 49.66% protection against neutrophil leakage, and 67.35% protection against protein leakage. DBCO(20×)-IgG liposomes, without any drug, altered the course of inflammatory lung injury to limit protein and leukocyte edema in the alveoli.

### Lysozyme-Dextran Nanogels Specifically Adhere in Injured Human Lungs Ex Vivo

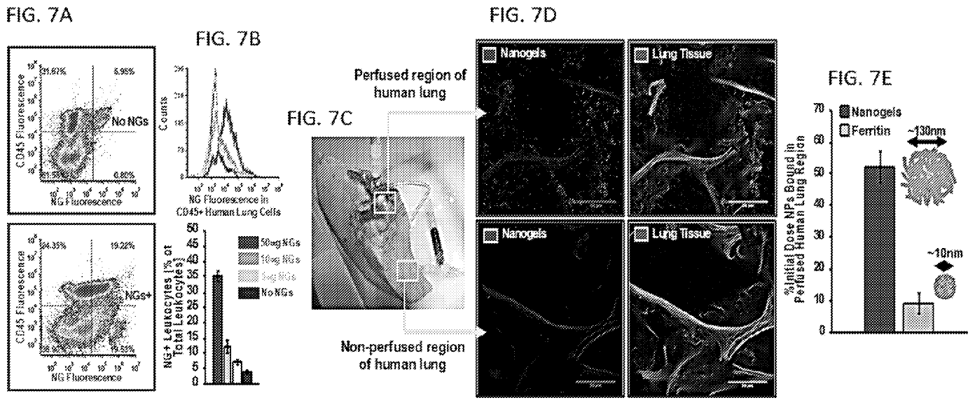

Fluorescent LDNGs were tested for targeting to single cell suspensions prepared from human lungs rejected for donation. 5 μg, 10 μg, or 50 μg of LDNGs were incubated with ˜10⁶ cells in suspension for 1 hour at room temperature. After three washes to remove unbound LDNGs, cells were stained for CD45 and analyzed with flow cytometry (FIG. 7A-7B). The majority of LDNG uptake in the single cell suspensions was attributable to CD45-positive cells. LDNGs accumulated in the human leukocytes, extracted from injured lungs, in a dose-dependent manner, with 35.08% of leukocytes containing LDNGs at a loading dose of 50 μg.

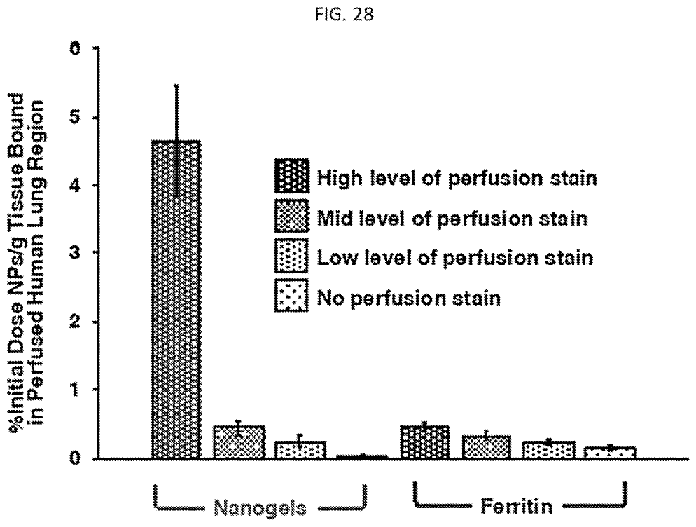

Finally, fluorescent or ¹²⁵I-labeled LDNGs were infused via arterial catheter into ex vivo human lungs excluded from organ donation due to conditions (including edema and injury) resembling those found in ARDS patients. Immediately prior to LDNG administration, tissue dye was infused via the same catheter to stain regions of the lungs directly perfused by the chosen branch of the pulmonary artery (FIG. 7C). After infusion of LDNGs, phosphate buffered saline infusion rinsed away unbound particles. Perfused regions of the lungs were dissected and divided into ˜1 g segments, divided into regions deemed to have high, medium, or low levels of staining with tissue dye. For lungs receiving fluorescent LDNGs, well-perfused and poorly-perfused regions were selected for sectioning and fluorescent imaging. Fluorescent signal from LDNGs was clearly detectable in sections of well-perfused tissue, but not poorly-perfused tissue (FIG. 7D). In experiments with ¹²⁵I-labeled LDNGs, ¹³¹I-labeled ferritin was concurrently infused (i.e. a mix of ferritin and LDNGs was infused) as an internal control particle shown to have no affinity for injured mouse lungs. For LDNGs and ferritin infused into the same lungs via the same branch of the pulmonary artery, LDNGs retained in the lungs at 52.15% initial dose and ferritin retained at 9.27% initial dose (FIG. 7E). LDNG adhesion in human lungs was focused in regions of the lungs with high levels of perfusion stain, with concentrations of 4.66% ID/g in the “high” perfusion regions, compared to 0.44% ID/g in the “medium” perfusion regions. Ferritin adhesion was more diffuse, with 0.47% ID/g in the “high” perfusion regions, compared to 0.35% ID/g in the “medium” perfusion regions (FIG. 28). Therefore LDNGs, a nanoparticle shown to accumulate in neutrophils in acutely inflamed lungs, avidly adhered in perfused regions of injured human lungs, but ferritin nanocages, a particle with no affinity for neutrophils in settings of inflammation, had only low levels of diffuse non-specific adhesion in injured human lungs.

### Example 2: Complement Mediated Uptake of Nanoparticles With Agglutinated Protein

Nanoparticles with agglutinated protein (NAPs) are a very broad class of nanoparticles that we have shown have tropism for neutrophils in animal models of ARDS, sepsis, and pneumonia. We sought out to identify the mechanism underlying nanoparticles uptake by neutrophils.

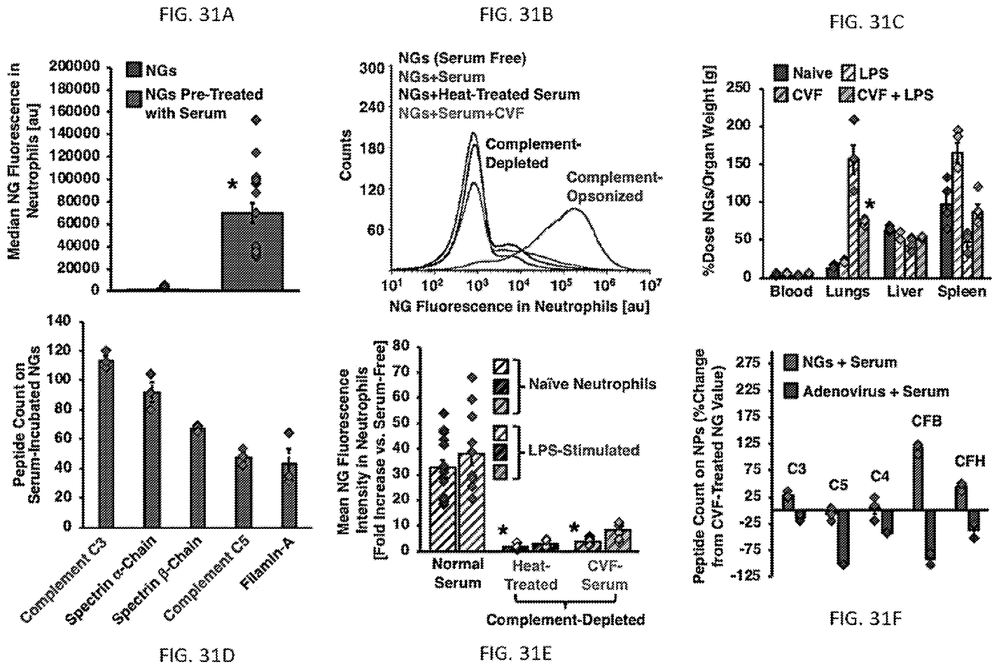

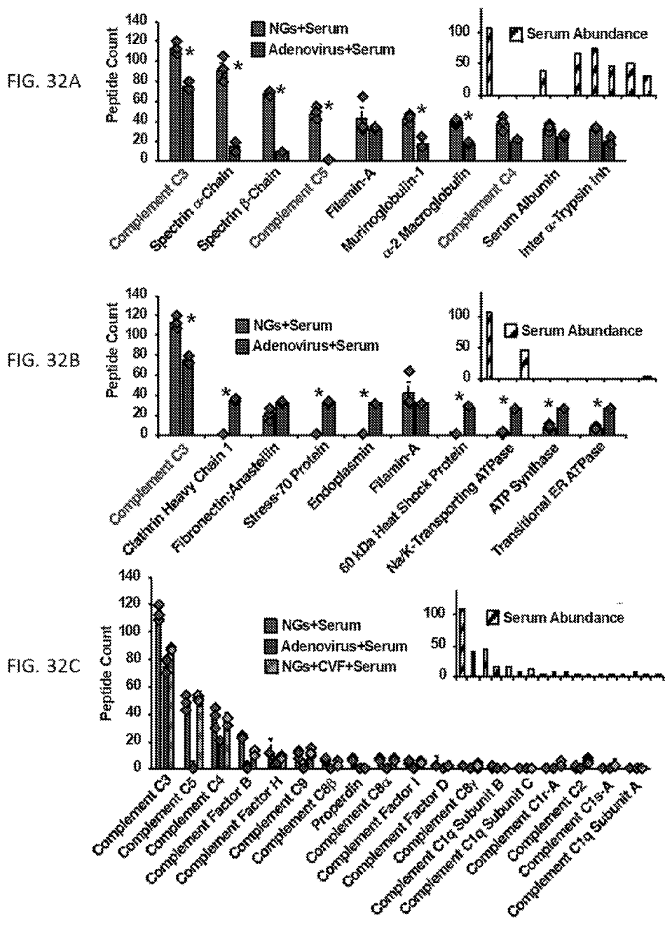

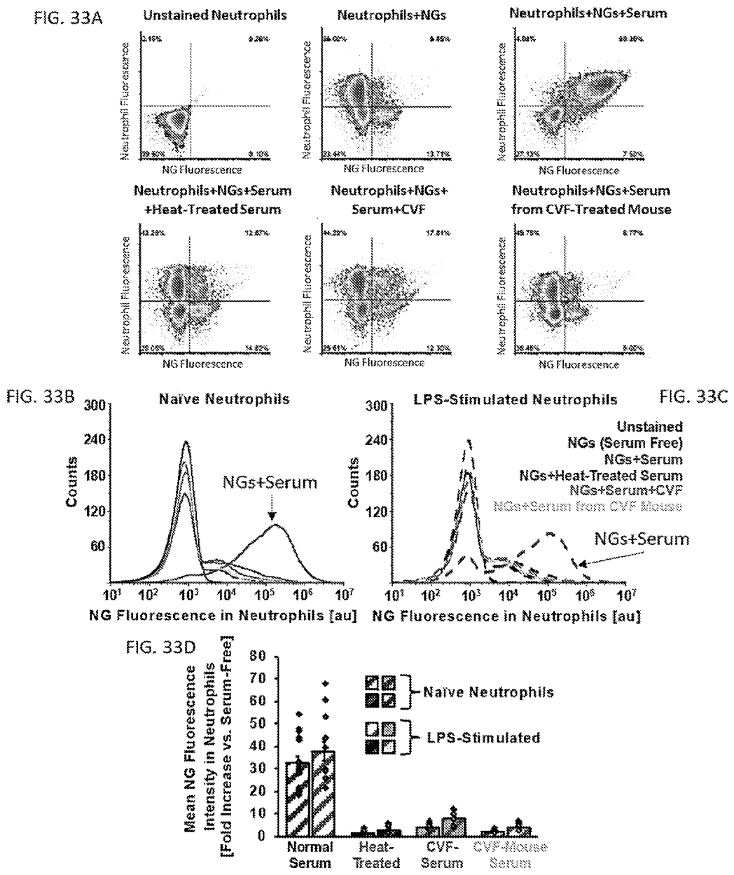

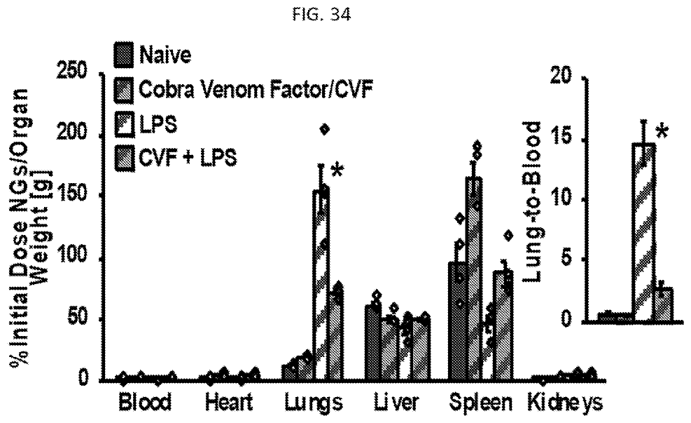

We first showed that neutrophils only take up nanoparticles in vitro if the NAPs were first exposed to serum (i.e. proteins dissolved in the non-cellular fraction of blood) (FIG. 31A-FIG. 31F). We then performed a proteomics analysis (using mass spectrometry) to identify the proteins that bind to NAPs (FIG. 32A-FIG. 32C). We found that complement proteins were the primary opsonins or serum proteins bound to the surface of particles. We found that eliminating complement proteins largely abrogated uptake of the nanoparticles into neutrophils, both in vitro and in vivo (FIG. 33A-FIG. 33D, FIG. 34). Notably, complement binding was much weaker to non-NAP nanoparticles, such as crystalline protein nanoparticles like adenovirus and AAV.

Our findings suggest that D20-tagged liposomes (see Example 1) cloak themselves in complement proteins to attain tropism for neutrophils. The D20-tagged liposomes act as a “decoy” for marginated neutrophils, causing the neutrophils to leave the lung and reducing their deleterious effects. Thus, we have identified a mechanism by which D20-tagged liposomes not only bind complement and have neutrophil tropism, but also ameliorate a severe ARDS model. Further, our findings indicate that the therapeutic efficacy of nanoparticles can be improved by generating or designing nanoparticles that are capable of binding to complement, thus improving their uptake by neutrophils.

### Example 3: Therapeutic Effects of D20-Tagged Liposomes

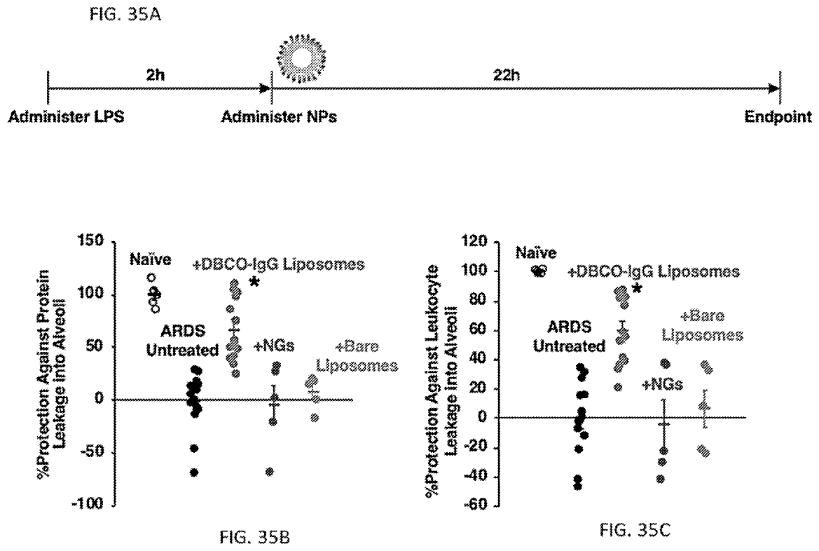

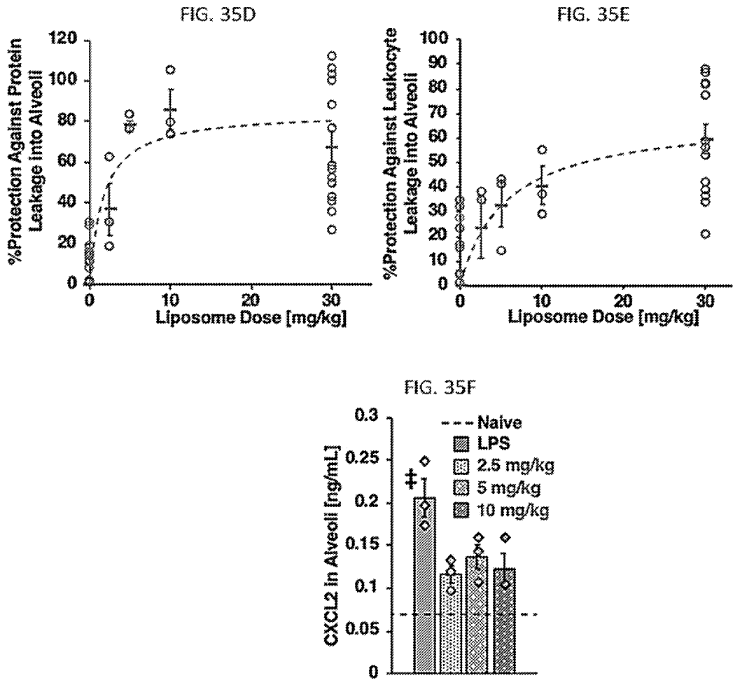

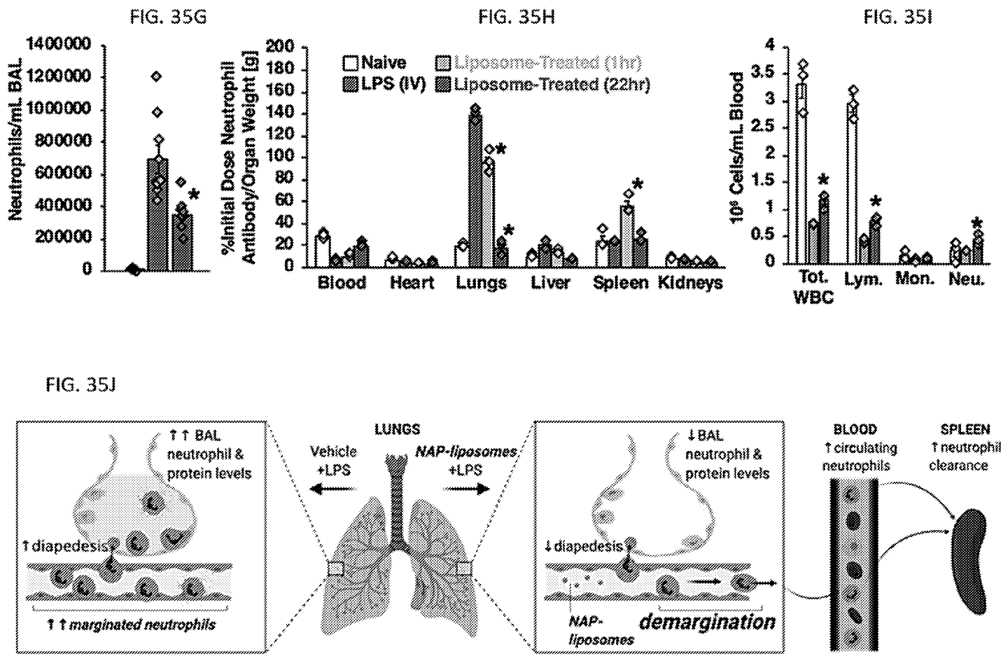

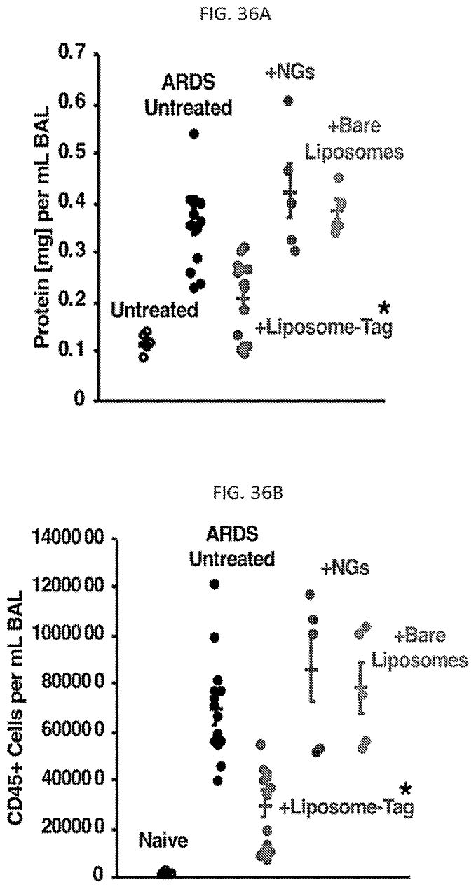

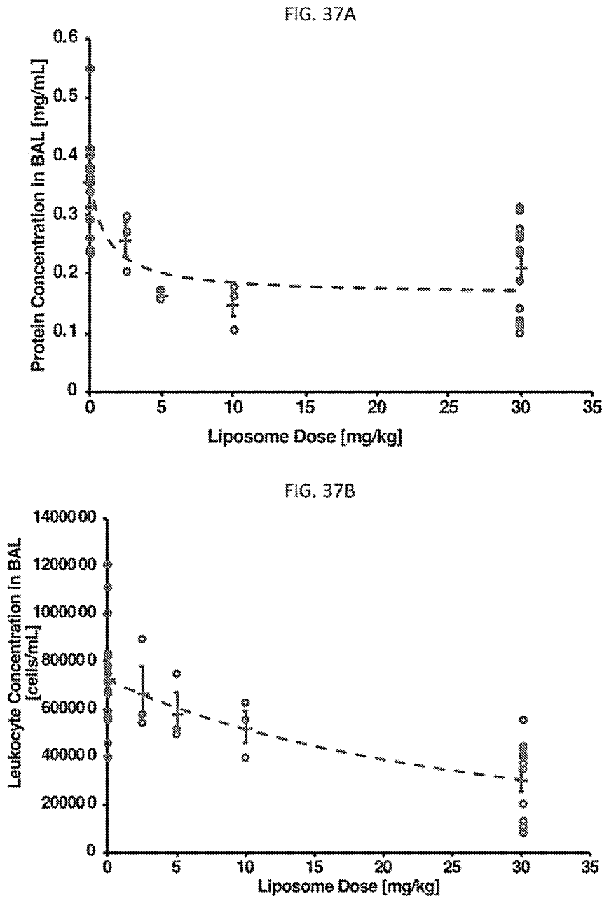

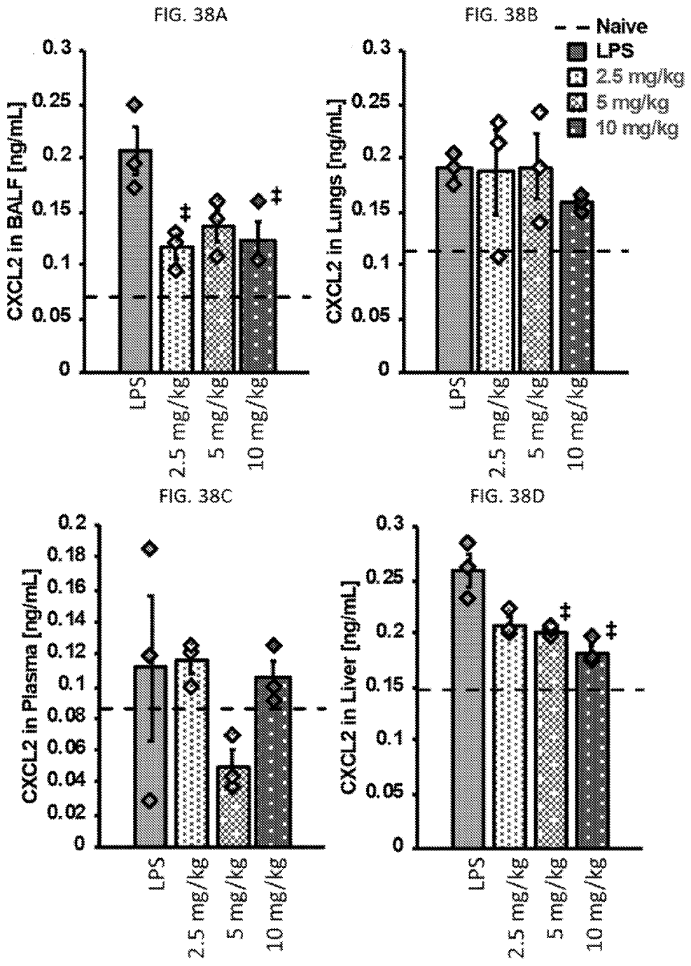

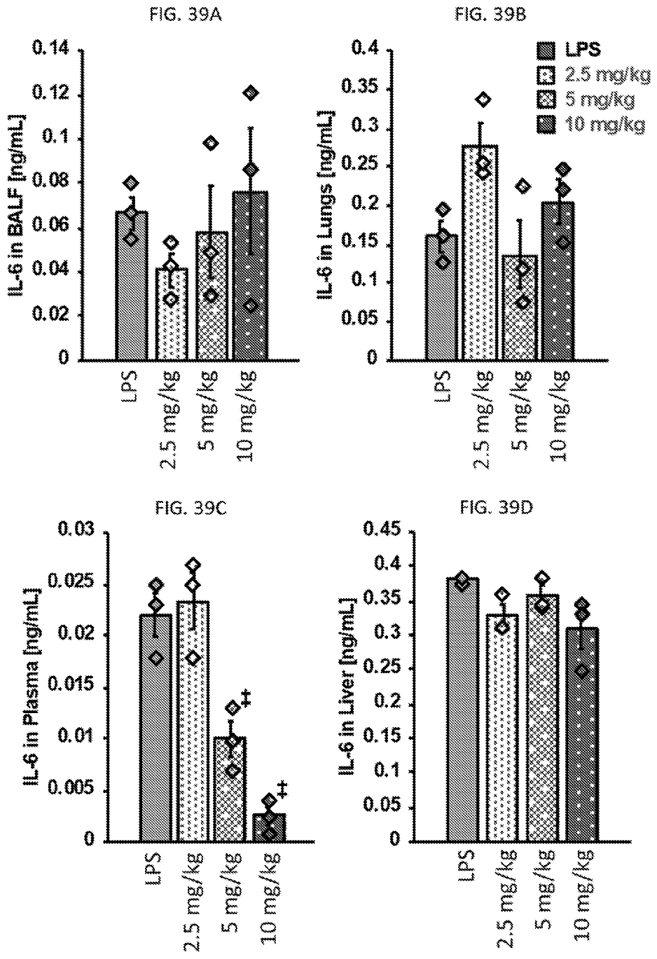

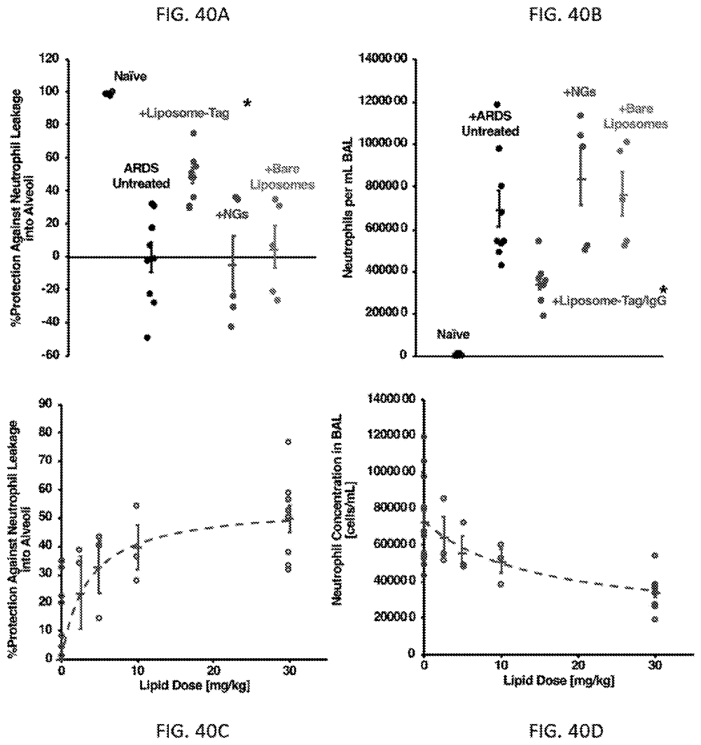

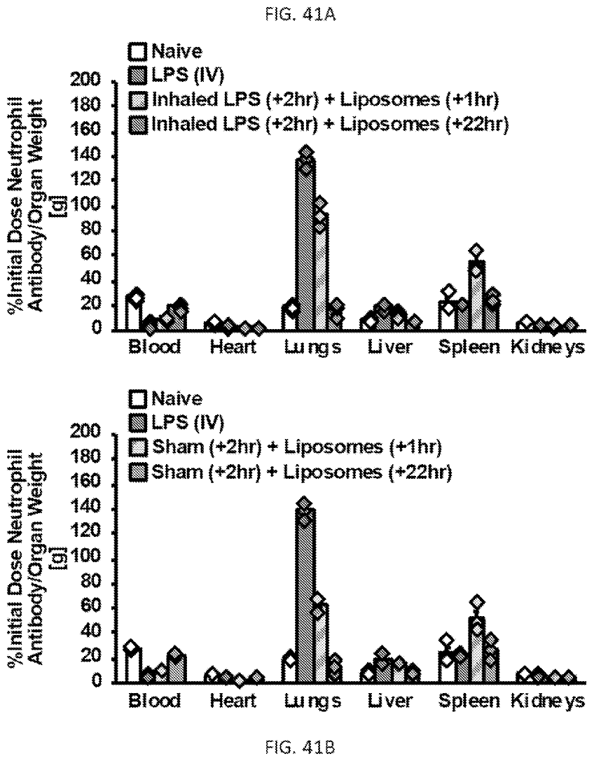

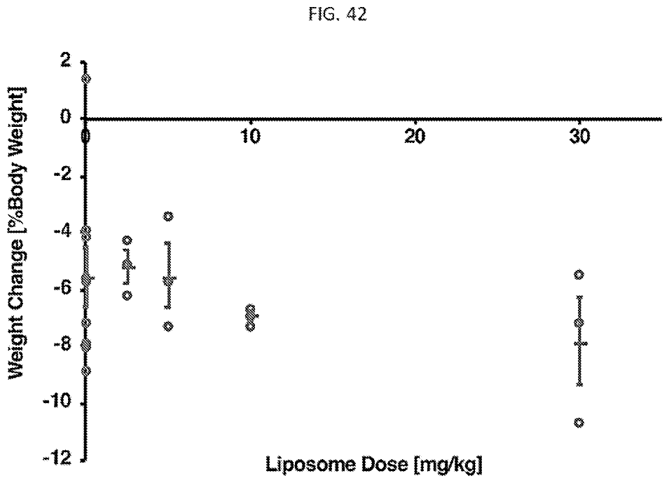

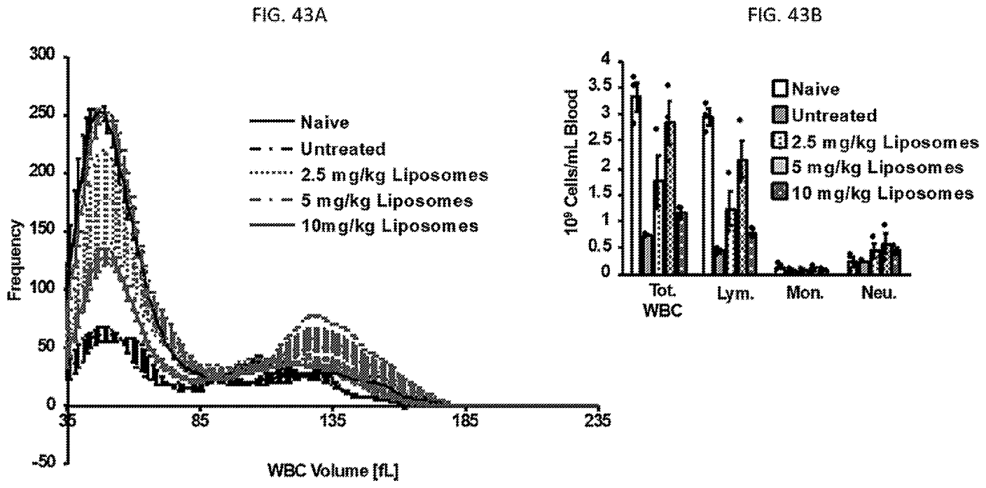

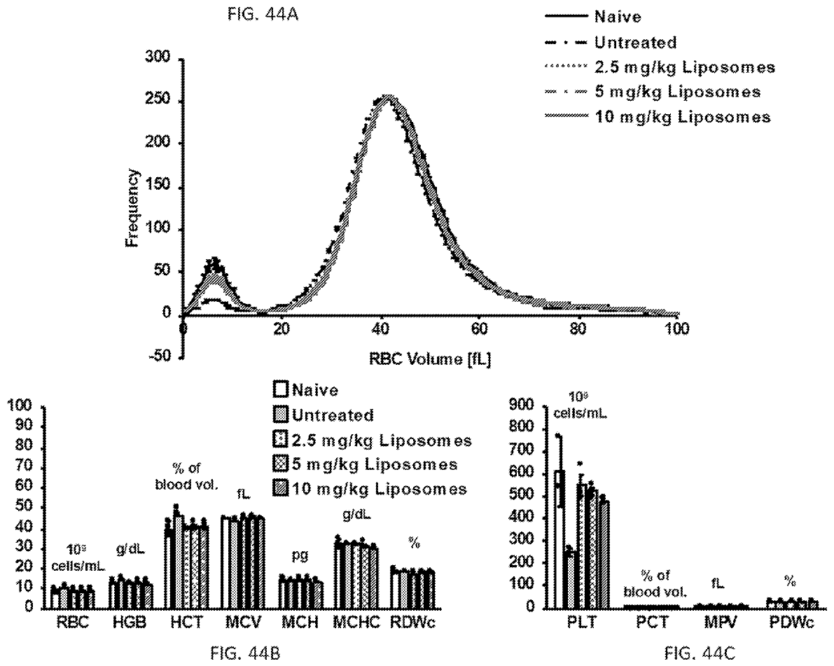

As described in Example 1, D20-tagged liposomes ameliorate a mouse model of severe acute respiratory distress syndrome (ARDS). We further investigated the therapeutic potential of D20-tagged liposomes for treatment of ARDS (FIG. 35A-FIG. 35J). For example, dose-response curves were generated (FIG. 35D and FIG. 35E), and we examined multiple additional ARDS phenotypes (including measuring cytokines in the lungs and blood) (FIG. 38A-FIG. 38D and FIG. 39A-FIG. 39D). As in Example 2, we found that the D20-tagged liposomes “cloak” themselves in complement, and thus appear to marginated neutrophils (a target cell essential to ARDS pathology) as if they are opsonized bacteria. In this manner, the D20-tagged liposomes act as decoys, and marginated neutrophils take up D20-tagged liposomes and leave the lung and migrate to the spleen, where they are known to undergo apoptosis (FIG. 35I). Thus, the D20-liposomes can be administered to target marginated neutrophils, which then migrate out of a site of injury or inflammation, instead of remaining and possibly causing further damage.

The results from these studies further demonstrate that D20-tagged liposomes can act as decoys that cause marginated neutrophils to leave the lungs and retire to the spleen. Marginated neutrophils in the lungs are major players in the pathology of ARDS, pneumonia, and sepsis, and thus D20-tagged liposomes can serve as a broadly applicable therapeutic.

### Example 4: Targeting Sites of Tissue Inflammation

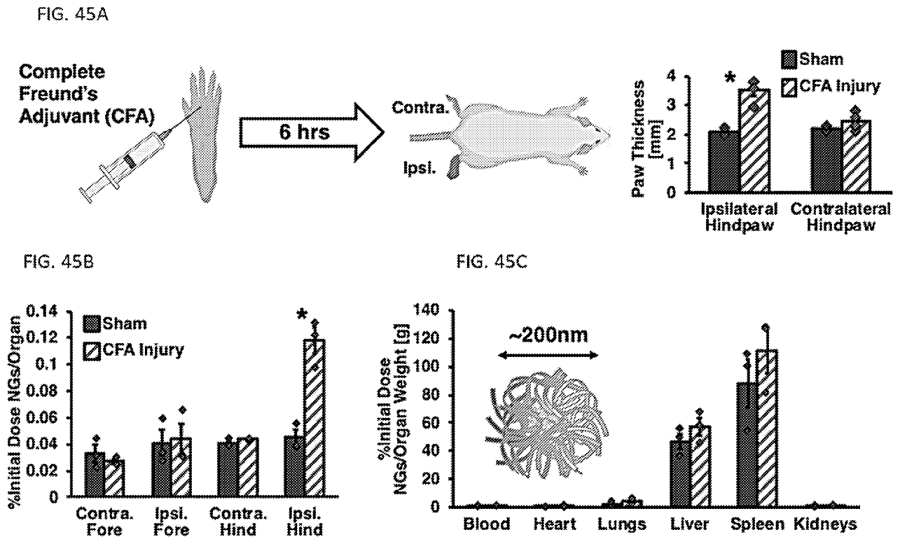

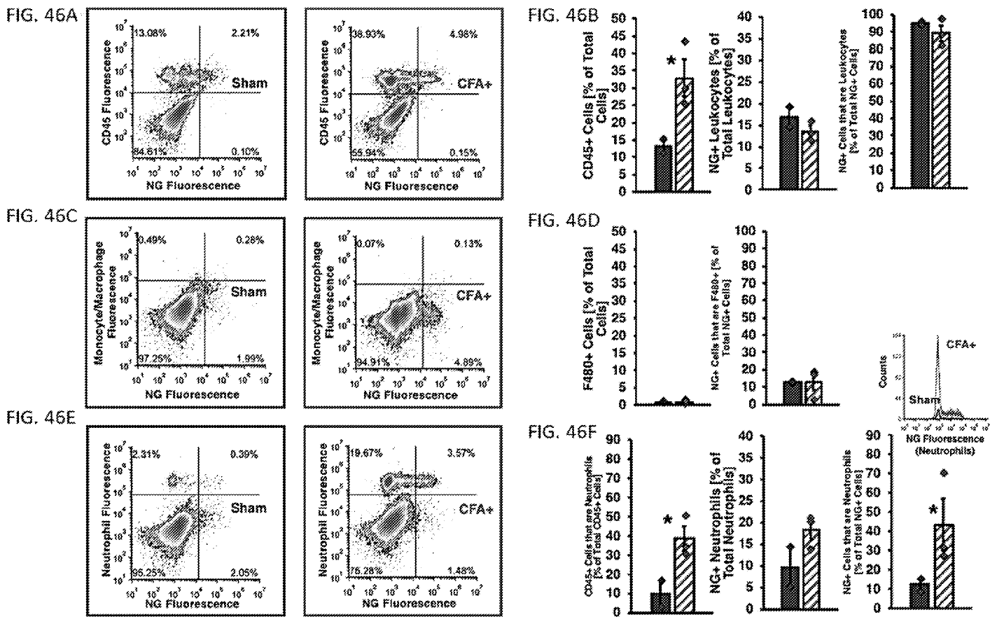

We had previously focused on targeting lung tissue. We sought to determine whether nanoparticles could be useful in additional contexts of inflammation as result of infection or injury in other tissues. We used a common model of subacute tissue inflammation, the injection of CFA (Complete Freund's adjuvant) into the footpad of a mouse (FIG. 45A-FIG. 45C). We showed that lysozyme-dextran nanogels localized to and were mostly taken up by neutrophils at the site of inflammation (FIG. 46A-FIG. 46F).

These findings extend the number of conditions where nanoparticles can be useful in addition to lung injury or inflammation. In particular, the results indicate that administration of D20-tagged liposomes would be beneficial for targeting a site of tissue inflammation in various disorders, including acute or subacute infections or inflammatory disorders.

All documents cited in this specification are incorporated herein by reference. U.S. Provisional Patent Application No. 62/943,469, filed Dec. 4, 2019, is incorporated herein by reference. While the invention has been described with reference to particular embodiments, it will be appreciated that modifications can be made without departing from the spirit of the invention. Such modifications are intended to fall within the scope of the appended claims.

1. A composition comprising a nanoparticle having a D20 tag comprising dibenzocyclooctyne (DBCO) covalently attached to a protein.

2. The composition according to claim 1, wherein the protein is an antibody or albumin.

3.-4 (canceled)

5. The composition according to claim 1, wherein at least 5, at least 10, at least 15, or at least 20 DBCO molecules are present per protein molecule.

6. The composition according to claim 1, wherein the nanoparticle is covalently attached to the D20 tag.

7. The composition according to claim 1, wherein the nanoparticle is a liposome, a lipid nanoparticle (LNP), or a protein-based nanoparticle.

8-9. (canceled)

10. The composition according to claim 1, further comprising a therapeutic molecule or diagnostic molecule loaded in the nanoparticle.

11. The composition according to claim 10, wherein the therapeutic molecule comprises an antimicrobial or a drug for treatment of acute respiratory distress syndrome (ARDS).

12. (canceled)

13. The composition according to claim 1, further comprising a targeting molecule conjugated to the nanoparticle.

14. The composition according to claim 13, wherein the targeting molecule is an antibody and is different from the protein of the D20 tag.

15. (canceled)

16. The composition according to claim 14, wherein the targeting molecule is an anti-PECAM antibody or anti-ICAM antibody.

17. A method of generating a composition comprising a nanoparticle having a D20 tag comprising dibenzocyclooctyne (DBCO) covalently attached to a protein, the method comprising conjugating DBCO to the protein to generate the D20 tag.

18. The method according to claim 17, further comprising covalently conjugating the D20 tag to the nanoparticle.

19. The method according to claim 17, wherein (a) the D20 tag is generated by reacting DBCO with the protein at a ratio of at least 5:1, or (b) the D20 tag is generated by reacting DBCO with the protein at a ratio of at least 20:1.

20. (canceled)

21. The method according to claim 17, further comprising
(a) contacting ex vivo the nanoparticle in a suspension with serum and/or or a solution containing complement proteins; and
(b) isolating and/or washing the nanoparticles.

22. A method of treating lung injury in a subject in need thereof, the method comprising administering the composition according to claim 1 to the subject.

23. The method according to claim 22, wherein the subject has ARDS, sepsis, pneumonia, or inflammatory lung injury.

24. (canceled)

25. The method according to claim 22, comprising administering the composition intravenously or intraarterially.

26.-27 (canceled)

28. A method of targeting leukocytes in a subject, comprising administering the composition according to claim 1 to the subject.

29. The method of claim 28, wherein the leukocytes are neutrophils, monocytes, macrophages, eosinophils, basophils, NK cells, lymphocytes, or dendritic cells and/or the leukocytes are marginated and/or present in the lung of the subject.

30. (canceled)

31. A method of treating an inflamed tissue in a subject in need thereof, the method comprising administering the composition according to claim 1 to the subject.

32-36. (canceled)
